(19) United States
(12) Patent Application Publication
(10) Pub. No.: US 2006/0246426 A1
(43) Pub. Date: Nov. 2, 2006

(54) Recombinant fusion proteins with high affinity binding to gold and applications thereof

(75) Inventors: Richard G. Woodbury, Seattle, WA (US); Theo deVos, Seattle, WA (US); Meher Irani, Edmonds, WA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: BIOHESION, INC., Seattle, WA (US)

(21) Appl. No.: 11/408,634

(22) Filed: Apr. 21, 2006

Related U.S. Application Data
(63) Continuation-in-part of application No. 10/671,995, filed on Sep. 26, 2003.

(51) Int. Cl. C12Q 1/70; C12Q 1/68; G01N 33/53; C12M 1/34

(52) U.S. Cl. 435/5; 435/6; 435/7.1; 435/287.2; 977/900

(57) ABSTRACT

The present invention provides a method to firmly attach any polypeptide to a gold surface regardless of its intrinsic gold-binding properties. The method describes the production of recombinant fusion proteins consisting of polypeptides of interest and a high affinity gold binding peptide consisting of 1 to 7 repeats of a unique amino acid sequence. By this method, many biologically active polypeptides lacking intrinsic gold-binding properties can be firmly attached to gold surfaces. The disclosure includes evidence that fusion proteins containing the gold-binding sequences provide superior stability and activity compared to similar molecules lacking the tag when used to construct biosensors. The invention provides a method that is a significant improvement over existing chemical and physical adsorption protocols to attach polypeptides to gold and, therefore, can provide benefits to many applications utilizing gold.

### RELATED APPLICATION

This application is a Continuation-in-Part of U.S. Ser. No. 10/671,995, filed Sep. 26, 2003, and claims benefit U.S. Provisional Application No. 60/675,405, filed on Apr. 28, 2005 and U.S. Provisional Application No. 60/681,349, filed May 16, 2005, the entire contents of each of which is incorporated herein by reference.

### STATEMENT OF GOVERNMENT SUPPORT

This invention was made in part with government support under Grant No. CA101579-01 R43 awarded by the National Cancer Institute, the National Institutes of Health. The government has certain rights in this invention.

### BACKGROUND OF THE INVENTION

1. Field of the Invention

The present invention relates generally to the production of fusion proteins and more specifically to production of recombinant fusion proteins for biosensors having gold binding proteins.

2. Background Information

Robust attachment of proteins and other macromolecules, e.g., recognition or affinity-binding molecules or enzymes, to a surface such as gold is an important step in implementing a variety of technologies targeting numerous applications in clinical diagnostics, laboratory research, biosensors, biomaterials, proteomics, and drug discovery/evaluation fields. Gold is an excellent material for introducing surface functionality via the attachment of proteins or other macromolecules because of the metal's chemical inertness, electrical conductivity, surface uniformity and stability, biologic compatibility/low toxicity and other properties. Gold's chemical inertness, however, limits the ability to prepare functional surfaces to just a few proteins or other macromolecules that produce stable biofilms when adsorbed directly onto a clean gold surface. For example, certain classes of immunoglobulin, streptavidin, protein A and certain proteins or peptides with basic charges passively adsorb to gold in appropriate buffers containing relatively low concentrations of salts at a certain pH range.

Many proteins and macromolecules of interest, however, do not adsorb readily to gold with subsequent retention of biological activity. Whether or not a particular molecule binds to gold depends on certain molecular properties and solvent conditions. Most important, the surface charge of proteins and other molecules appears to affect the interaction with gold, favoring those molecules with basic charges. Therefore, the current methods of direct adsorption of proteins and other macromolecules to gold are successful only for a relatively few examples of the large number of molecules of interest with commercial potential. The method of direct adsorption of molecules to gold, therefore, severely impedes the development of novel applications in all fields utilizing gold.

Most of the current methods for direct physical adsorption of proteins, other macromolecules and small molecules to gold result in complexes that are unstable. For example, complexes of immunoglobulin or protein A and gold can dissociate in aqueous solution prior to, during, and following intended applications or can be displaced from gold in the presence of other proteins and macromolecules during applications. Such instability can lead to inconsistent results for test samples, limit the number of potential applications, and result in gold-protein complexes that have short storage lives.

Further, direct adsorption of proteins, other macromolecules, and small molecules to gold can be a random process in regard to which surface of the molecule binds gold. Random attachment can result in inefficient orientation or presentation of active sites of molecules that interact with target molecules or substrates in solution. Improper orientation of active sites on a significant proportion of molecules on gold can reduce the sensitivity and utility of molecule-gold complexes in applications.

Also, direct adsorption of macromolecules (especially proteins) to gold frequently results in molecular denaturation or inactivation when molecules in solution bind directly to such surfaces. Denaturation of proteins, in particular, can lead to waste of valuable proteins and can increase non-specific binding of materials to the surface causing fouling.

Moreover, it seems that only large molecules such as proteins, proteoglycans, or structures such as membrane-bound lipids typically bind well to gold. With the exception of sulfur-containing compounds and certain salts and other ions, most small molecules have weak affinity to gold. Consequently, many small polypeptides including hormones, antigens, steroid-based hormones, other receptor ligands, pesticides, other environmental toxins, or the like cannot be attached directly to gold. Methods exist for the covalent attachment of desired small molecules linked via reactive groups in foundation layers of bovine serum albumin or thiol compounds that can bind gold. Such approaches, however, are inefficient for the general reasons discussed above for proteins. In addition, small molecules of interest typically contain few or no suitable reactive groups for attachment to foundation layers and many small molecules are inactive following covalent attachment to a foundation layer.

The general ineffectiveness of current methods for direct adsorption of proteins and other macromolecules to gold as described above has stimulated effort to develop improved methods for introducing active molecules to gold surfaces. In one process, alkanethiol monolayers with reactive groups at the distal end of the molecules can be introduced on gold to allow attachment of molecules of interest at the surface. In this manner, the desired molecules typically do not interact directly with the gold surface. Further, such biofilms can be unstable in complex solutions or whenever sulfur-containing compounds are present. Moreover, these biofilms have limited utility in applications outside of the laboratory.

In the field of surface plasmon resonance (SPR) biosensors, in particular, BIAcore (Sweden) achieved improvements in the stability and utility of alkanethiol monolayers on gold through the covalent attachment of a layer of high molecular weight dextran to the monolayer (Jonsson, et al., BioTechniques 11:620-627, 1991). The dextran hydrogel contains reactive groups for attaching proteins and other macromolecules in a favorable hydrophilic environment. The introduction of the dextran layer also stabilizes the alkanethiol monolayer on gold and helps reduce non-specific binding to gold. The BIAcore technology supports commercial instruments used entirely for research purposes where test conditions can be strictly controlled. However, analysis of complex clinical and environmental samples remains problematic for BIAcore's instruments because the sulfur-gold linkage is labile when samples contain sulfur-based compounds, including proteins with surface cysteines. Additionally, while BlAcore's technology reduces non-specific binding during testing of simple, well-defined laboratory solutions, non-specific binding precludes testing of many environmental, clinical, industrial and other complex samples with BlAcore instruments.

The discovery of a gold-binding peptide, GBP, and studies by Woodbury and coworkers (Woodbury, et al., Sensors & Bioelectronics, 13:1117-1126, 1998) led to a chemical method to link recognition proteins to gold via GBP to construct SPR biosensors (U.S. Pat. No. 6,239,255). The process requires binding a recombinant GBP-alkaline phosphatase chimera to the gold surface, removing the alkaline phosphatase domain with proteases, activating chemical groups on the GBP domain that remains attached to gold, and introducing the desired recognition protein for covalent attachment to the GBP foundation. However, the process is tedious, inefficient and not readily applicable to constructing arrays consisting of many different proteins or other macromolecules that can require numerous, different chemical procedures to achieve attachment of all molecules of interest. Further, the approach can have limited usefulness for applications utilizing colloidal gold, which can be unstable under certain conditions required for the covalent attachment of molecules to reactive groups on GBP or other foundation layers.

In the case of molecules available in minute quantities, conventional methods can fail to attach sufficient numbers of molecules to gold. Increasingly, advances in nanotechnology and array technology require greater control of molecular orientation of nanomolar/picomolar amounts of material than is possible using current attachment chemistries. Novel applications utilizing colloidal gold can be developed, for example, if the relatively few molecules that bind to this form of gold can be extended to any protein, other macromolecules, and small polypeptides and other molecules of interest. In fact, certain bio-detection platforms use colloidal gold or nano gold particles derivatized with bioactive molecules as biosensors in many applications. For example, derivatized colloidal gold is a basic component in lateral flow test strips also known as immunochromatographic strips (ICS), where such devices are used as in vitro diagnostics (IVD).

Colloidal gold (CG)—typically 20 nm, 40 nm, and somewhat larger sized particles—is used extensively as a detection component in IVD test kits. CG is used also in research as a contrast material in electron microscopy. The term nano gold (NG) has been used to refer to CG, but increasingly nano gold is used to describe particles that are 1 to just a few nm in diameter. Nano gold provides superior results in electron microscopy, and can be attached to certain proteins to increase their electrical conductivity. Existing methods to derivatize CG or NG with proteins generally depend on physical adsorption. This process can be inefficient and is limited to those proteins that have affinity to CG or NG. Further, many small peptides of interest such as antigenic peptides cannot be readily adsorbed to CG or NG. Further, such conventional methods can result in CG-/NG-protein complexes that become unstable during storage or use.

Various reactive groups can be attached to certain forms of NG—usually through a sulfur-Au linkage—to which proteins can be covalently attached. Attachment of polypeptides to surfaces require reactive specific amino acids (e.g., lysine, glutamate, histidine and others) or on the amino or carboxy termini. The idiosyncratic nature of polypeptides precludes general application and the use of a specific chemical method can produce variable success for different polypeptides. Additionally, where chemistry is dependent on modification of specific amino acids, the chemistry itself may destroy biological activity of polypeptides. Further, coupling reactions can require harsh solvent or conditions that can destroy biological activity of polypeptides, including that sulfur-Au linking chemistry to derivatize gold can destabilize other linkages when sulfides, sulfhydryls, or other sulfur containing compounds are present in test samples.

For other bio-detection platforms, the catalysis of a substrate by an enzyme can provide the basis for a quantitative assay to measure the substrate concentration when the chemical event is translated to a signal that is detectable: e.g., enzyme electrodes. Enzyme electrodes were first used as biosensors to measure oxygen. The coupling of enzyme activity to an electrical signal has been an active area of research for many years. The use of gold is pervasive in electronic testing and measuring devices because its chemical resistance, electrical conductivity, and other properties, make gold an excellent bio-detection surface. Enzyme electrode biosensors have enormous potential in medical diagnostic, industrial, and environmental testing. An example of a significant commercial success is glucose oxidase (GOx)-based monitors for home use to measure blood glucose levels in diabetics.

Sensitivity and selectivity are two important factors that determine whether or not a practical enzyme electrode can be constructed. Higher applied potential and concentration of electron transfer mediator, and close proximity of enzyme to electrode can enhance biosensor sensitivity. On the other hand, too high an applied potential will result in the oxidation of irrelevant substances in samples and selectivity will suffer. A critical balance of conditions, therefore, must be determined for each distinct enzyme electrode. An ideal situation would be a mediator-free system operating at very low applied potential. Such electrodes, however, require very high sensitivity.

### SUMMARY OF THE INVENTION

The present invention discloses a method to achieve robust, efficient immobilization of any polypeptide to the surface of colloidal or nano gold particles regardless of the intrinsic capacity of the polypeptide to bind gold directly. The invention can be applied to fabricate devices designed for clinical and environmental diagnostic testing, and industrial applications. The present invention can greatly expand the number of potential testing applications that are based on colloidal or nano gold complexes with specific polypeptides. The invention discloses recombinant fusion proteins capable of derivatizing CG or NG with any desired polypeptide. This is accomplished by including a gold-binding peptide (GBP) fusion partner in the recombinant proteins. Appropriate conditions allow selective binding of GBP to GC or NG while minimizing surface interaction with polypeptide fusion partners. Fusion partners, e.g., enzymes can be tethered from the gold surface into solution with retention of up to 100% of activity.

Further, the invention can be applied to the construction of enzyme electrodes capable of quantitative measurement of specific analytes in clinical and environmental diagnostic testing and in many industrial applications. The invention discloses recombinant fusion proteins capable of introducing any desired enzyme activity to gold electrodes. This is accomplished by including a gold-binding peptide (GBP) fusion partner in recombinant proteins. Appropriate conditions are used to optimize selective binding of GBP to gold while minimizing surface interaction with other fusion partners. Fusion partners, e.g., enzymes are tethered from the surface into solution with retention of up to 100% of activity. Controlling molecular orientation of enzymes and other proteins is an important goal for surface chemistry to enhance sensitivity of devices using CG/NG and enzyme electrode biosensors.

In one embodiment, a device for analyte detection is disclosed including a carrier and a first gold-comprising solid phase having a first immobilized fusion protein thereon having at least one gold binding protein (GBP) domain and at least one analyte-binding peptide (ABPP) or analyte-binding protein (ABP) domain, where the GBP domain includes SEQ ID NO:1, and where the at least one ABPP or ABP is reactive with one or more analytes.

In one aspect, the first gold-comprising solid phase is a plurality of mobilizable colloidal-gold particles, nano-gold particles, or gold-coated particles comprising a first region on the carrier. In a related aspect, a second region on the carrier is disclosed, where the second region comprises at least one immobilized moiety which binds to the at least one ABPP, ABP, analyte, or complexes thereof. In a further related aspect, the carrier includes a first member adapted for drawing a deposited sample from the first region of the carrier to the second region of the carrier, where the plurality of mobilizable particles comprise a sample application area and the at least one immobilized moiety comprises a capture zone.

In another aspect, the first gold-comprising solid phase is a first gold electrode. In a related aspect, a second electrode is included, where the first electrode is a working electrode and the second electrode is a reference electrode. In a further related aspect, the ABPP or ABP functions as a molecular transducer in the absence of a mediator.

In one aspect, the carrier comprises a surface opposing the first electrode, and where the opposing surface comprises a separate immobilized ABPP or ABP.

In another aspect, a signal is generated upon the reaction of the ABPP or ABP and the analyte via a gain or loss of electrons from the electrode, where the gain or loss of electrons comprises a current flowing in a circuit connected to the first electrode upon the reaction of the ABPP or ABP and the analyte. In a related aspect, a third electrode is included and a first circuit electrically connecting the second and third electrodes for producing a predetermined potential on one of the second and third electrodes, and a second circuit attached to the first electrode where a current is produced in the second circuit connected to the first electrode when the ABPP or ABP reacts with the analyte in order to produce a signal proportionate to the concentration of the analyte in a sample. In a further related aspect, the signal is a change in potential, which is measured by a change in impedance.

In another embodiment, a method of detecting an analyte is disclosed including exposing a device to a sample, an analyte-containing environment, or an analyte containing-surface, where the device comprises, a carrier, and a first gold-comprising solid phase having a first immobilized fusion protein thereon including at least one gold binding protein (GBP) domain and at least one analyte-binding peptide (ABPP) or analyte-binding protein (ABP) domain, where the GBP domain includes SEQ ID NO:1, and detecting the interaction between the at least one ABPP or ABP and the analyte.

In one aspect, the first gold-comprising solid phase is a plurality of mobilizable colloidal-gold particles, nano-gold particles, or gold-coated particles comprising a first region on the carrier. In a related aspect, the method includes allowing the sample to interact with the mobilizable particles, immobilizing the particles in at least one capture zone of a second region, and detecting the presence or absence of the analyte in the at least one capture zone. In a further related aspect, detecting includes identifying a pattern which is a function of the presence or absence of the analyte, where the pattern is detected visually, microscopically, or spectroscopically.

In another aspect, the first gold-comprising solid phase is a first gold electrode, and detection includes measuring a signal generated from the interaction, where the signal is generated upon the reaction of the ABPP or ABP and the analyte via a gain or loss of electrons from the electrode, and wherein the gain or loss of electrons comprises a current flowing in a circuit connected to the first electrode upon the reaction of the ABPP or ABP and the analyte.

In a related aspect, the device further includes a second electrode, where the first electrode is a working electrode and the second electrode is a reference electrode. In a further related aspect, the device includes a third electrode and a first circuit electrically connecting the second and third electrodes for producing a predetermined potential on one of the second and third electrodes, and a second circuit attached to the first electrode whereby a current is produced in the second circuit connected to the first electrode when the ABPP or ABP reacts with the analyte in order to produce a signal proportionate to the concentration of the analyte in a sample.

A further understanding of the nature and advantages of the invention will become apparent from the detailed description, other specific examples of the invention, and other information provided below.

### BRIEF DESCRIPTION OF THE DRAWINGS

FIG. 1 depicts a general scheme for constructing GBP fusion proteins with any polypeptide partner(s) whereby the GBP sequence is positioned at the amino terminus, internally, or at the carboxyl terminus of the recombinant molecule. The drawings represent the DNA sequence encoding the fusion protein portion of a plasmid vector that can be expressed in host cells.

FIG. 2 depicts the plasmid map of the expression vector, pPA-GBP, designed to produce His₆-protein A-GBP fusion protein in E. coli cells.

FIG. 3 depicts the plasmid map of the expression vector, pStreptavidin-GBP, designed to produce His₆-streptavidin-GBP fusion protein in E. coli cells.

FIG. 4 depicts the SDS-PAGE analyses of the production of recombinant proteins, His₆-protein A-GBP, His₆-streptavidin-GBP-, and His₆-streptavidin in E. coli cells. Panel A, BHI-4 and Panel B BHI-7 and BHI-9 expression. Panel A, Protein A-GBP: lanes: 1, MW standards; 2, soluble fraction, non-induced; 3, soluble fraction, induced; 4, insoluble fraction, non-induced; 5, insoluble fraction, induced. Panel B: left side, soluble fraction; right side, insoluble fraction; lanes: center, MW standards; 1 & 8, induced streptavidin-GBP; 2 & 9, non-induced streptavidin-GBP; 3 & 10, non-expressing vector control; 4 & 11, induced streptavidin; 5 & 12, non-induced streptavidin; 6 & 13, non-expressing vector control. Arrows (←) indicate position of fusion proteins.

FIG. 5 depicts the SDS-PAGE analyses of the purification of recombinant proteins, His₆-protein A-GBP, His₆-streptavidin-GBP-, and His₆-streptavidin from cell extracts facilitated via the His₆ tag binding to nickel resin columns. Panel A, protein A-GBP. Lanes: 1, soluble fraction, induced; 2, insoluble fraction, induced; 3, insoluble fraction, non-induced; 4-9 Ni++ resin eluate fractions. Panel B, Lanes: 1-4, SA-GBP eluate fractions; 5-8, SA eluate fraction; 9, MW standards.

FIG. 6 depicts the selective cleavage of protein A-GBP fusion protein at an inserted Asn-Gly bond. Hydrolysis of Protein A-GBP at an inserted Asn-Gly bond by hydroxylamine at pH 9.5. Lanes: 1, 3, & 5, no hydroxylamine; 2, 4, & 6, 2M hydroxylamine; 1-4, addition of 4M urea; 5 & 6, no urea; 1 & 2, overnight incubation at 42° C.; 3-6, 4 hour incubation at 42° C.; 7, MW standards.

FIG. 7 depicts the gold binding and antibody binding activities of His₆-protein A-GBP fusion protein on gold powder compared to these activities of native protein A on gold powder.

FIG. 8 depicts the gold binding and biotin-binding activities of His₆-streptavidin-GBP fusion protein and recombinant His₆-streptavidin (lacking the GBP domain) on gold powder.

FIG. 9 depicts how gold stabilizes the GBP domain of His₆-streptavidin-GBP in the presence of guanidine-HCl. Black: before binding to gold; White, after binding to gold.

FIG. 10 depicts sensorgrams of analyses of SPR biosensors constructed with His₆-protein A-GBP fusion protein or native protein A. SPR analysis of individual sensors constructed with native protein A (gray line) and protein A-GBP (black line). Raw data results displayed superimposed for comparison. Analysis on native protein A sensor approximately 5-fold higher resolution than that of protein A-GBP. RI, refractive index. Indicated by arrows: 1, 3, and 5, PBS/BSA; 2, mouse IgG; 4, goat anti-mouse antibody. Antibodies diluted 1:1000 in PBS/BSA.

FIG. 11 depicts sensorgrams of analyses of SPR biosensors constructed with recombinant His₆-streptavidin-GBP or His₆-streptavidin. SPR analysis of individual sensors constructed with recombinant sreptavidin (gray line) or sreptavidin-GBP fusion (black line). Raw data results displayed superimposed for comparison. Downward drift (gray line) may indicate slight loss of adsorbed protein from sensor during analysis. RI, refractive index. Indicated by arrows: 1, 3, and 5, PBS/BSA; biotinylated anti-mouse IgG; 4, mouse IgG conjugated with alkaline phosphatase. Antibodies diluted 1:1000 in PBS/BSA. Rapid increase in RI after 4 is due to glycerol in antibody preparation.

FIG. 12 depicts the plasmid map of the expression vector, pPA-GBP-PA, designed to produce His₆-protein A-GBP-protein A fusion protein in E. coli cells.

FIG. 13 depicts the plasmid map of the expression vector, pStrept-GBP-Strept, designed to produce His₆-streptavidin-GBP-streptavidin fusion protein in E. coli cells.

FIG. 14 depicts the plasmid map of the expression vector, pPA-GBP-Streptavidin, designed to produce His₆-protein A-GBP-streptavidin fusion protein in E. coli cells.

FIG. 15 depicts the plasmid map of the expression vector, pStreptavidin-GBP-PA, designed to produce His₆-streptavidin-GBP-PA fusion protein in E. coli cells.

FIG. 16 depicts the plasmid map of the expression vector, pGBP, designed to produce His₆-GBP (GBP monomer) fusion protein in E. coli cells.

FIG. 17 depicts the plasmid map of the expression vector, pGBP-GBP, designed to produce His₆-GBP-GBP (GBP dimer) fusion protein in E. coli cells.

FIG. 18 depicts a GBP-fusion protein bound to a gold surface. In this representation, the GBP sequence is fused to a single-chain antibody partner. The design of this system results in complete accessibility of analyte molecules, e.g., antigens, to the binding site of the GBP-fusion partner.

FIG. 19 depicts the binding facilitated binding of GBP-fusion proteins to CG or nanogold particles compared to conventional binding of polypeptides.

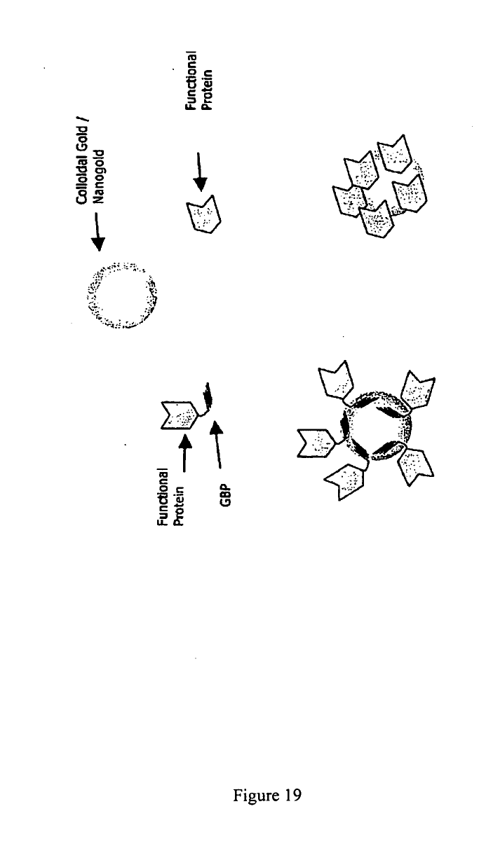

FIG. 20 depicts the example of enhanced IVD test sensitivity that can be achieved by including HRP or similar enzyme in GBP-fusion proteins.

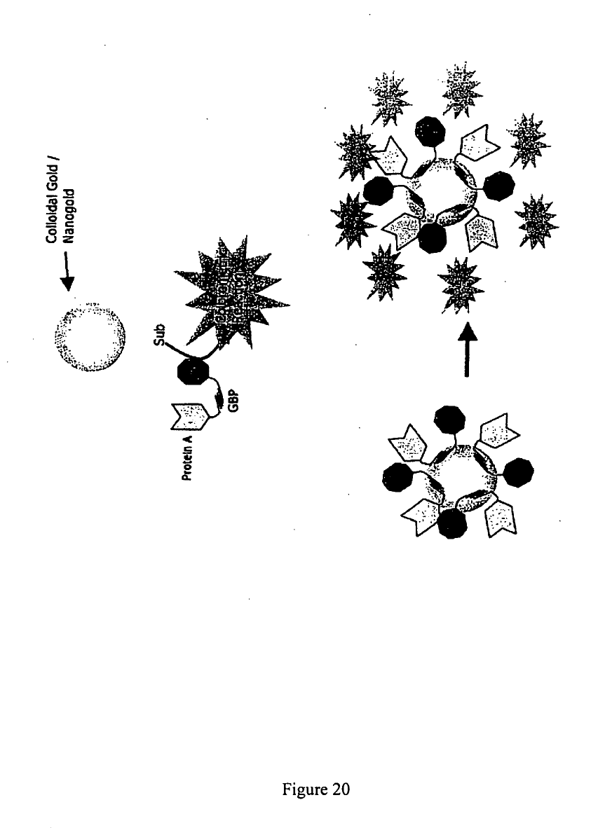

FIG. 21 depicts a coupled enzyme electrode system to measure glucose concentration.

FIG. 22a illustrates the inventive concept to couple GOx and HRP activities on a gold electrode as a molecular complex.

FIG. 22b illustrates the inventive concept to couple GOx and HRP activities on a gold electrode as a single recombinant protein molecule.

FIG. 23 depicts the plasmid map of the expression vector, pStreptavidin-GBP-HRP, designed to produce Streptavidin-GBP-horseradish peroxidase fusion protein.

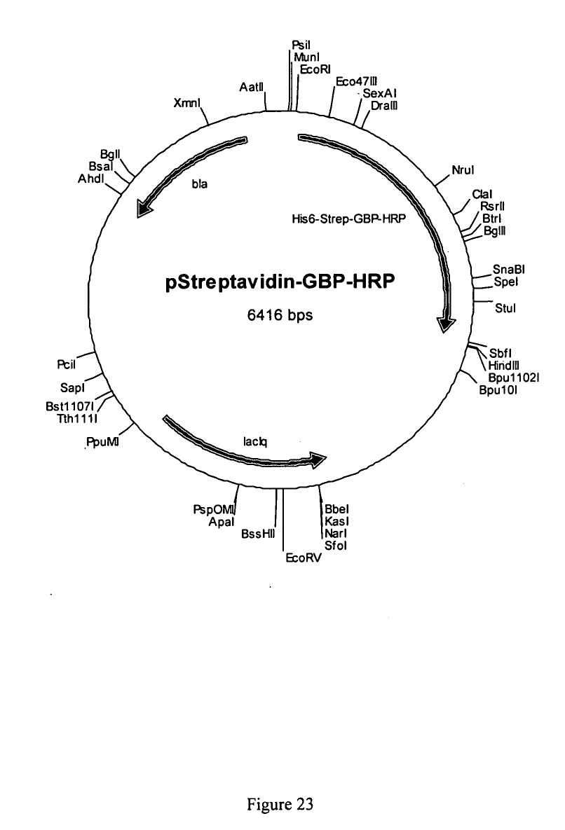

### DETAILED DESCRIPTION OF THE INVENTION

Before the present composition, methods, and treatment methodology are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

The term “carrier,” including grammatical variations thereof, as used herein means a relatively inert solid phase of matter upon which other materials may be placed. For example, such material can include, but is not limited to, absorbent surfaces, and non-conducting materials, such as glass, ceramics, or non-conducting polymers.

The term “gold-comprising solid phase,” including grammatical variations thereof, as used herein means a phase of matter characterized by resistance to deformation and to changes of volume that contain, comprise, or are coated with the element gold.

The term “analyte binding peptide (ABPP),” including grammatical variations thereof, as used herein means an amino acid sequence which may be less than a full length protein or gene product that has the ability to interact with an analyte.

The term “analyte binding protein (ABP),” including grammatical variations thereof, as used herein means a substantially full length protein or gene product that has the ability to interact with an analyte.

In a related aspect, an ABPP or ABP can include, but is not limited to, protein A, protein G, streptavidin, core streptavidin, neutravidin, avidin, avidin related protein 4/5, strep-tag, strep-tag II, an antibody, an antibody fragment, a single chain antibody, a protein antigen, a peptide antigen, a peptide toxin, biotin, an enzyme, a receptor, a peptide ligand, a polypeptide substrate, a polypeptide inhibitor, or combinations thereof.

The term “mobilizable,” including grammatical variations thereof, as used herein means having the ability to be released from one position in space or on a carrier to another position in space or on a carrier. For example, beads adsorbed on a wick are mobilizable, and change position on the wick when suspended in a fluid.

The term “immobilized moiety,” including grammatical variations thereof, as used herein means a membrane bound compartment, chemical, mixture of chemicals, or mixture of molecules that are limited in their freedom of movement when such a compartment, chemical or mixture of chemicals are adsorbed on a solid phase. In a related aspect, an immobilized moiety includes, but is not limited to, peptide, a polypeptide, an organic molecule, an inorganic molecule, a nucleic acid, a lipid, a carbohydrate, a prokaryotic cell, a eukaryotic cell, a virus, or a combination thereof.

The term “capture zone,” including grammatical variations thereof, as used herein means a region on a surface where movement is limited by the interaction of an immobilized moiety and an analyte, ABPP, ABP, or complex comprising a combination thereof.

The term “detectable pattern,” including grammatical variations thereof, as used herein means a discernable configuration, the existence of which can be determined, discovered, or measured.

In a related aspect, the detection of the configuration may be accomplished by spectroscopic methods, which includes detection by any instrument which analyzes a continuum of wavelengths especially, for example, in the visible region of the electromagnetic spectrum.

The term “electrode,” including grammatical variations thereof, as used herein means a conductor used to establish electrical contact with a nonmetallic part of a circuit, including elements in a semiconductor device (as a transistor) that emits or collects electrons or holes or controls their movements. In a related aspect, a “working electrode” is the electrode where the potential is controlled and where the current is measured. In another related aspect, the “reference electrode” is used in measuring the working electrode potential.

The term “mediator,” including grammatical variations thereof, as used herein means molecules which can shuttle electrons between the redox center of an enzyme and an electrode. In a related aspect, the present invention avoids the use of mediators by direct electron transfer through biocatalytic transduction.

The term “substrate,” when referring to catalytic activity, means a substance acted upon by the active site of an enzyme.

The term “impedance” as used herein means the apparent opposition in an electrical circuit to the flow of an alternating current that is analogous to the actual electrical resistance to a direct current and that is the ratio of effective electromotive force to the effective current.

The term “potential” as used herein means the work required to move a unit positive charge from a reference point (as at infinity) to a point in question.

The term “low complexity” as used herein means a few in number of different sequences.

The invention described herein produces recombinant fusion proteins consisting of a unique GBP consisting of 7 repeats of the 14 amino acid sequence, Met-His-Gly-Lys-Thr-Gln-Ala-Thr-Ser-Gly-Thr-Ile-Gln-Ser (SEQ ID NO:1), and any desired polypeptide specifying activity, binding such fusion protein to a gold surface thereby introducing functionality to the surface.

The use of gold is pervasive in electronic testing and measuring devices because its chemical resistance, electrical conductivity, and other properties, make gold an excellent bio-detection surface. GBP has been shown to be useful to derivatize the gold surface of SPR sensors for bio-detection (Woodbury, R G, et. al., Biosensors and Bioelectronics, 13:117-1126, 1998; Furlong and Woodbury, U.S. Pat. No. 6,239,255 B1).

Redox enzymes, in particular, offer much potential for the design of biosensors because excellent electronic communication can occur following substrate catalysis. Enzyme electrodes can be either amperometric or potentiometric in nature. Amperometric sensors consist of working and reference electrodes. Applied potential at the working electrode at a fixed value relative to a reference electrode allows signal transduction of enzyme activity in the form of electron transfer between solution and electrode. In the case of an amperometric enzyme electrode the observed current is proportional to the substrate concentration when an electroactive species is generated during catalysis.

The simplified enzymatic reaction is:

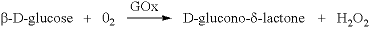

The redox center of GOx lies deep within the enzyme molecule and direct electronic communication with the electrode does not occur. To overcome this, electronic transfer mediators such as hydroquinone, p-benzoquinone, or ferrocene are added to facilitate signal transduction.

A large variety of potentiometric enzyme electrodes are also possible. Electrodes of this type differ from amperometric devices in that the information from enzyme activity is converted into a potential signal. The signal is logarithmically proportional to the concentration of a particular analyte. Potentiometric devices rely on selective electrodes that can be highly specific for analytes. Compared to amperometric electrodes, conventional potentiometric devices can be less sensitive and responsive.

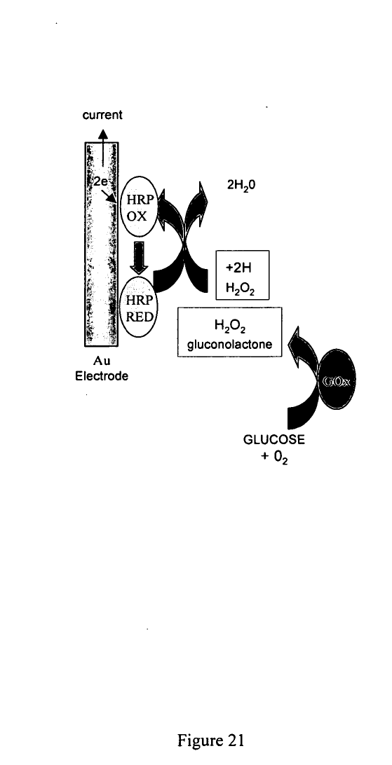

Many investigators have observed that significantly enhanced signal transduction is possible by also including, e.g., horseradish peroxidase (HRP) that uses the GOx reaction product hydrogen peroxide as substrate. Generally, electron transfer mediators are also required to achieve adequate sensitivity at low applied potential. A schematic representation of the coupled enzyme system is shown in FIG. 21.

The capacity to directly couple enzyme activity with the capability of electronic devices is a highly coveted goal, given the enormous array of applications possible if this goal is achieved. In contrast to methods to immobilize nucleic acids on surfaces, the complexity of protein chemistry presents substantially greater challenges for enzymes. A major barrier to the routine development of commercial enzyme electrode biosensors is the absence of a predictable, more utilitarian method for attaching any enzyme of interest to electrodes.

An essential step in constructing an enzyme electrode is the immobilization, entrapment, or compartmentalization of the enzyme on or near an electrode to facilitate electrochemical communication. While many enzyme electrodes work well in the laboratory under highly controlled conditions and in relatively simple solutions, there are serious challenges in producing commercial devices. Real-world testing is difficult because sample compositions and properties are variable and hard to control. Some of the difficulties encountered in testing biological and environmental samples are instability of the recognition enzyme element, low sensitivity, high background interference, and electrode fouling. Some of these problems, e.g., the presence of interfering substances are sample related. But many of the others are caused by inadequate immobilization or containment of enzyme.

Typically, the first step in constructing an enzyme electrode is accomplished by one of several methods: physical adsorption, covalent attachment, affinity capture, containment, or entrapment of enzyme on or near the working electrode.

Physical adsorption is the prevalent methodology for fabricating enzyme electrodes, and usually the method of choice for proteins in general, e.g., as in ELISA testing. It is commonly used for nucleic acid chemistry where electrostatic adsorption provides for robust binding. For enzymes, the approach is sufficient when the amount of enzyme is not limiting and activity is retained. Many potentially useful enzymes, however, do not bind surfaces well, lose activity when they do bind, or are only weakly bound following adsorption. Certain glycoproteins, including enzymes such as horseradish peroxidase, bind poorly to gold. Also, nearly all enzymes denature to varying degrees upon surface contact. Further, there is little control over molecular orientation. Enzyme molecules randomly bind surfaces and substrate access to enzyme catalytic sites can be hindered.

The present invention describes the process to fabricate superior enzyme electrode biosensors compared to conventional methods. Enzymes are fused to GBP to allow binding of active enzymes to gold electrodes.

Electron transfer between native HRP and electrode is slow and inefficient. This is mainly attributed to the poor and random binding of glycosylated HRP to electrodes. In recent years, considerable effort has been made to improve the binding of HRP to gold. Genetically engineered variants of the enzyme expressed in E. coli have improved binding to gold electrodes. A similar recombinant HRP lacking carbohydrate when adsorbed to gold electrodes was capable of direct electron transfer without the requirement of electron transfer mediators.

Covalent attachment chemistries are available for the linking of enzymes to surfaces, based on reactivity of specific amino acids (e.g., lysine, glutamate, histidine and others) or on the amino or carboxy termini. Frequently, a reactive foundation layer must be introduced on the surface to attach enzymes. Foundation layers may introduce additional problems, such as durability, background interference, and decreased electrode conductivity. The idiosyncratic nature of enzyme properties precludes general application, since the use of a specific chemical method can produce variable success for different proteins. In addition, where chemistry is dependent on modification of specific amino acids, the chemistry itself may destroy enzyme activity. Further, coupling reactions can require harsh solvent or extreme conditions that may inactivate enzymes or adversely affect cofactors.

Affinity capture methods have been developed using surface attached proteins such as Streptavidin/Avidin to bind enzyme-biotin conjugates. This approach can provide stable attached enzymes, but attachment of Streptavidin directly to surfaces or to foundation layers has the same constraints as described above.

Containment requires enzymes to be concentrated near electrodes and partitioned from test solutions with semi-permeable membranes. This approach may have the advantage of preventing direct contact of interfering macromolecules to electrodes, but may suffer insensitivity due to poor electrochemical communication.

Entrapment of recognition enzymes into electro compatible materials that form films or sol-gel layers directly on electrodes is common. Such composite electrodes may provide increased sensitivity and enzyme stability. The fabrication process can be complex, expensive and high quality control may be difficult to achieve. Also, some enzymes may be inactivated during the entrapment procedure.

In contrast, no linking chemistry is required to attach desired polypeptides to GBP. With conventional methods different coupling chemistries can be required to attach distinct proteins to a GBP or other foundation layer. For example, when protein array chips are constructed with hundreds or thousands of unique proteins the complexity of many different linking chemistries, variable reaction rates and unequal protein coupling present formidable challenges to achieve functional uniformity on any single array and consistency among replicate arrays. The recombinant molecules provided by the present invention eliminate these technical difficulties and uncertainties by simplifying the entire surface derivatization process to a single, rapid step, i.e., the specific interaction of GBP and gold. Thus, in a related aspect a method is provided to achieve high uniformity and consistency in the manufacture of gold chips, colloidal gold, or any gold surface consisting of one or many distinct recognition or binding polypeptides or enzymes.

In one embodiment, the invention encodes a gold-binding peptide (GBP) for the stable attachment of fusion proteins to any gold surface. In a related aspect, a second component includes, but is not limited to, a fusion partner consisting of any desired polypeptide with specific binding or enzyme activity. For example, the inclusion of short, flexible amino acid sequences of low complexity linking GBP and fusion partner domains facilitates optimum physical orientation of each domain to allow full expression of GBP and fusion partner activities. In another related aspect, a third component including, but not limited to, a specific polypeptide affinity tag, e.g., polyhistidine (His₆-tag), permits rapid purification of the fusion protein in essentially one step. Rapid purification from cellular extracts or secretions can minimize proteolytic degradation typically associated with the expression of fusion proteins. In one aspect, the presence of the affinity tag in fusion proteins, obviates the need for each fusion protein to require a separate purification scheme.

In another aspect, the disclosed method allows for the attachment of proteins and small polypeptides to gold by transferring the gold-binding process to a polypeptide domain designed for this purpose (i.e., GBP). Further, the invention provides a rapid, one-step purification procedure that can be used for all fusion proteins of the type disclosed.

In one aspect, such fusion proteins include, but are not limited to, specific chemical or enzyme cleavage sites in the linking amino acid sequences between domains to allow the physical separation of fusion partner domains.

In one embodiment, plasmid expression systems in bacterial, yeast, insect, and mammalian cell lines for the production of fusion proteins whereby GBP is placed at the amino terminus, internally, or at the carboxyl terminus of any other polypeptide are disclosed. In a related aspect, fusion partners of GBP include, but are not limited to, protein A, protein G, streptavidin, core streptavidin, neutravidin, avidin, avidin related protein 4/5, strep-tag, strep-tag II, an antibody, an antibody fragment, a single chain antibody, a protein antigen, a peptide antigen, a peptide toxin, biotin, an enzyme, a receptor, a peptide ligand, a polypeptide substrate, a polypeptide inhibitor, metallothionein, receptors, or any other affinity binding polypeptide. Further, fusion partners include polypeptides that possess high affinity to bacteria or secreted products of bacteria and the like. Fusion partners also include polypeptides that have high affinity to viruses or parasites. In one aspect, fusion partners include small polypeptide hormones such as insulin or angiotensin, or vasoactive or neuroactive molecules that interact with receptors. In a further related aspect, small polypeptide fusion partners include, but are not limited to, peptide epitopes recognized by specific antibodies.

In one embodiment, affinity binding molecules of interest that are not polypeptides, which include, but are not limited to, nucleic acids, carbohydrates, lipids, lectins, and small molecules, which can be attached to the fusion protein on gold via one or more fusion partners. Specific nucleic acids, for example, can be labeled with biotin and can subsequently bind with high affinity to fusion proteins containing streptavidin. Similarly, fusion partners can be polypeptides that bind other cofactors or small molecules, where the cofactors and small molecules are linked to non-polypeptide targets.

In another embodiment, GBP-fusion partners can be polypeptides derived from screening, for example, diverse phage libraries, for active molecules. Active polypeptides can include those with selective binding affinity to specific proteins, or other macromolecules, small organic or inorganic molecules, surfaces other than gold, cells, viruses, parasites, or any substance of interest.

In one embodiment, the invention provides for one or two copies of cell surface receptor or protein such as a GPI-anchored fusion partner for each GBP domain. The fusion protein with two copies of GPI-linked protein provides an excellent model to study the binding process of ligands that normally occurs on the surface of cells whereby ligand cross-links two GPI-linked proteins to initiate a cellular function.

In another embodiment, the invention provides a GBP-fusion protein whereby the two fusion partners are distinct GPI-linked proteins or the stable binding domain of a different type of cell surface receptor.

In another embodiment, the invention provides a GBP-fusion protein whereby the fusion partner is one or more polypeptide ligands of a cell surface receptor or other macromolecule.

The production of fusion proteins containing certain enzymes and GBP provides a method to bind enzymes to gold with retention of optimal enzyme activity and other properties as generally described in this invention for any protein of interest. Currently, certain enzymes support applications in clinical testing, research, and industry generating total annual revenues of billions of dollars. These rapidly growing markets include glucose monitoring for diabetics, $3 billion/year; industrial enzyme use, $2 billion/year; and hundreds of millions of dollars annually for research enzymes. New monitoring devices intended for home use now under development, e.g., for cholesterol testing, will generate even larger markets.

he trends to smaller (nanotechnology), less expensive testing devices for home monitoring and research instruments requires innovative solutions to improve the efficiency of enzyme-based and other types of assays to support these devices. In particular, there is great demand for non-invasive or minimally invasive monitoring procedures. For example, if testing sensitivity can be increased above that of existing devices, many clinical tests can be developed to test salvia rather than blood. Also, the availability of more sensitive, low cost testing devices will facilitate the development of new monitoring approaches designed for home management of chronic diseases where daily testing would be beneficial. The invention disclosed, herein, will facilitate the fabrication of nanodevices in many fields because of several efficiencies (as previously described) that the technology of controlled orientation attachment of protein provides compared to existing methods.

GBP-containing fusion proteins can be produced that contain any two different enzymes, or one enzyme and a single-chain antibody, or one enzyme and any polypeptide with affinity for the substrate or product of the enzyme fusion partner.

In one embodiment, the different GBP-fusion partners can be horseradish peroxidase or related peroxidase and any oxidative enzyme. An advantage of such fusion proteins is to couple the electron-enhancing function of HRP and the like to the activity of any oxidative enzyme used to detect certain analytes.

In another embodiment, the different GBP-fusion partners can be any two enzymes or enzyme complexes with distinct activities that occur as coupled enzyme systems in nature. An advantage of such fusion proteins is to significantly enhance the overall activity of coupled enzyme systems whereby, the product of one enzyme is the substrate of the other. The close physical proximity of the two enzymes on a gold surface favors utilization of the concentrated product of the first enzyme by the second enzyme before the product can diffuse into the surrounding solution.

In another embodiment, the different GBP-fusion partners can be any two enzymes or enzyme complexes with distinct activities that do not occur as coupled enzyme systems in nature. An advantage of such fusion proteins is to provide a mechanism by which enzymes that naturally occur in uncoupled systems can be physically connected to each other and a gold surface. This allows the concentrated product of the first enzyme to be utilized as substrate of the second enzyme before the product can diffuse into the surrounding solution.

In another embodiment, the different GBP-fusion partners can be any enzyme and a scFv antibody with affinity binding activity for the substrate or product of the enzyme. An advantage of such fusion proteins is to provide a mechanism to concentrate any molecule of interest at a gold surface by binding the molecule to the fusion protein via a scFv with specificity to that molecule. A minor change in solvent conditions, e.g., increasing the salt concentration or changing the pH, can be used to release the concentrated molecule from the scFv antibody allowing the enzyme fusion partner to use the molecule as substrate. Alternatively, scFv antibodies can be selected that have relatively high dissociation constants, e.g., 10⁴ to 10⁻⁶ M, that function to concentrate the molecule of interest from dilute solution, but with low avidity to permit relatively rapid dissociation of the molecule and allow the enzyme to utilize it as substrate.

In one aspect, a GBP-fusion protein can have a scFv fusion partner that has specificity for Clostridium botulinum toxin A.

In another aspect, GBP-fusion proteins can have scFv fusion partners that in combination have specificity for six other serotypes of Clostridium botulinum toxin.

In another aspect, a GBP-fusion protein can have a scFv fusion partner that has specificity for the toxin, ricin.

In another aspect, a GBP-fusion protein can have a scFv fusion partner that has specificity for enterotoxin B from Staphylococcus aureus.

In another aspect, GBP-fusion proteins can have scFv fusion partners that have specificity for of the Category A-D list of toxins and agents for biowarfare.

In other aspect, GBP-fusion proteins can have scFv fusion partners with specificity to any toxin or poisonous agent.

In another aspect, a GBP-fusion protein can have a scFv fusion partner that has specificity for anthrax spores.

In other aspect, GBP-fusion proteins can have scFv fusion partners with specificity to any infectious agent.

In other aspect, GBP-fusion proteins can have scFv fusion partners with specificity to important clinical targets, e.g. to the drug digoxin.

In other aspect, GBP-fusion proteins can have scFv fusion partners with specificity to important environmental targets.

In other aspects, two identical copies of a specific scFv can be fused to a single domain of GBP to provide increased analyte-binding capacity to a given area of gold surface. This can increase the sensitivity of the signal out-put of biodetection instruments during testing.

In another aspect, the different GBP-fusion partners can be scFv antibodies with distinct specificity such as scFv1-GBP-scFv2. One advantage of such a fusion protein is to provide a means to concentrate two distinct molecules with interactive or reactive potential. This can be especially beneficial in cases where the dilute concentrations of two or more molecules in solution preclude their interactive or reactive potential.

In one embodiment, the invention discloses the production of a recombinant molecule containing GBP, a polypeptide binding a specific molecule A, and an enzyme utilizing molecule A as substrate. Such a molecule can concentrate molecule A in dilute solutions in the vicinity of the enzyme to allow a reaction not possible when all components are free in solution.

In another embodiment, the invention discloses a method for the production of a recombinant protein consisting of the enzyme horseradish peroxidase (HRP) fused to GBP. Many biological processes of interest generate peroxide that can provide the basis of clinical testing. Nature provides enzymes, i.e., peroxidases, to destroy cytotoxic peroxide. The electrons formed by peroxidase activity can produce an electrical current at a nearby electrode. High sensitivity can be achieved in assays of certain redox reactions using HRP fused to GBP to construct biosensing electrodes. For examples, the invention can be used to construct amperometric enzyme electrodes or other devices for the detection of hydrogen peroxide, organic hydroperoxides, phenols, aromatic amines and hazardous compounds, e.g., potassium cyanide.

As disclosed, recombinant HRP-GBP fusion protein, providing controlled orientation attachment of HRP to gold electrodes can offer increased sensitivity, less electrical resistance, and greater durability to redox-based amperometric sensing and other types of electrochemical detection devices.

In another embodiment, two molecules of HRP can be produced fused to a single domain of GBP. Such a fusion protein can have greater specific activity than a recombinant molecule with only one copy of enzyme.

Streptavidin is a non-glycosylated protein from Streptomyces avidinii that assembles as a tetrameric protein. It can non-covalently bind four molecules of D-biotin with a dissociation constant of 10⁻¹⁵ M. This rapid and almost irreversible binding has made Streptavidin a useful protein for the detection and characterization of various biological substances. Any desired enzyme can be biotinylated and then attached to streptavidin-GBP on a gold electrode to produce an enzyme-based biosensor when the activity of the enzyme changes the electron communication to the electrode.

Streptavidin-GBP-HRP can be used to construct biosensors using various combinations of HRP-coupled enzyme systems to measure different analytes when appropriate biotinylated enzymes are bound to Streptavidin when the biotinylated enzymes produce hydrogen peroxide in the presence of specific analytes.

In one embodiment, the disclosure provides a method to produce a recombinant protein containing the enzyme glucose oxidase (GOx) fused to GBP. The invention, herein, provides benefits in the design of miniature glucose monitoring devices or other devices incorporating nanotechnology by attaching glucose oxidase to a gold surface in an efficient, low cost method providing full retention of enzyme activity and other properties.

In another embodiment, two molecules of GOx can be produced fused to a single domain of GBP. Such a fusion protein can have greater specific activity than a recombinant molecule with only one copy of enzyme.

In one embodiment, fusion partners of GBP can be enzymes, for example, but not limited to, such as oxidases, oxidoreductases, hydrolases, esterases, dehydrogenases, including horseradish peroxidase (HRP), glucose oxidase (GOx), choline esterase, and cholesterol oxidase. For example, glucose oxidase or horseradish peroxidase may be used to construct monitoring devices to measure blood glucose levels in diabetics or other analytes.

A specific example of how such a coupled enzyme system can be achieved consists of attaching biotinyl-glucose oxidase (GOx) to streptavidin-GBP-HRP on gold electrodes to make a biosensor. GOx is the principle enzyme used in monitors to measure blood glucose levels in diabetics in the home (a market in excess of $3 billion/yr). Coupled with HRP activity to enhance the transduction signal due to GOx activity the present invention can increase sensitivity of measuring glucose concentration in samples. With so large a market size, even incremental improvements in overall testing performance can result in significant market share.

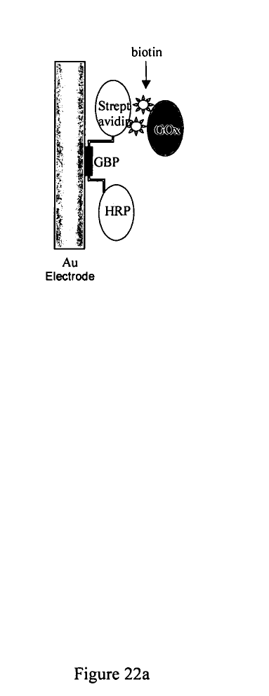

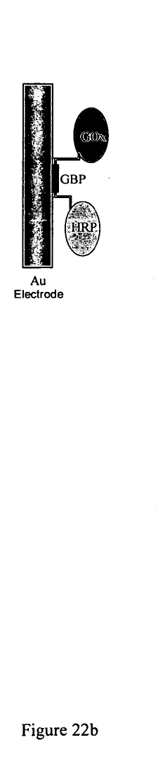

A general schematic representation of our invention on gold electrodes is depicted in FIG. 22a. Streptavidin-GBP-HRP can enable the fabrication of robust commercial enzyme electrode biosensors capable of supporting specific testing applications by substituting an appropriate biotinyl-enzyme for biotinyl-GOx.

The present invention describes a single recombinant fusion protein containing GOx and HRP attached to GBP and attached to gold electrodes as depicted in FIG. 22b. The invention can significantly reduce effort, time, and cost to construct a coupled GOx/HRP enzyme electrode biosensor. The invention also can enhance the overall performance of existing GOx/HRP coupled enzyme electrodes.

Cholesterol oxidase attached to an electrode can constitute an enzyme electrode bisoensor capable of measuring total blood cholesterol levels.

In one embodiment, the activity of cholesterol oxidase can be coupled to that of HRP to enhance overall sensitivity of enzyme electrodes that measure total cholesterol. Biotinylated cholesterol oxidase can be attached to Streptavidin-GBP-HRP on gold electrodes or cholesterol oxidase can be included as a fusion partner in the recombinant protein cholesterol oxidase-GBP-HRP or HRP-GBP-cholesterol oxidase.

In one aspect, fusion partners can be attached at either end of the GBP domain. Thus, methods are disclosed which permit two or more copies of a desired fusion partner attached to a single GBP domain to increase the specific binding capacity or enzymatic activity of the fusion protein attached to gold. For example, multiple copies of fusion partners can be expressed in tandem. In a related aspect, a minimum of two copies of a fusion partner can be expressed by placing one at the amino-terminus and the other at the carboxy-terminus of a single GBP domain.

In one embodiment, a method of producing fusion proteins containing two or more distinct fusion partners with different activities is disclosed. For example, a chimera can be produced containing streptavidin at one end of GBP and Protein A at the other end. In a related aspect, a fusion protein with multiple function is one containing two distinct enzymes attached to GBP. In another aspect, a mixed-function fusion protein is one whereby one fusion partner, e.g., a single-chain antibody or receptor, can bind specific molecules present in low concentration. The increased concentration of specific molecules in the vicinity of the fusion protein can significantly improve the activity of a second fusion partner, e.g., an enzyme that utilizes the specific molecules as substrate when conditions are changed to release the specific molecules from the binding domain of the fusion protein.

In one aspect, multiple and mixed function fusion proteins can have utility when applied to clinical diagnostic testing, or “lab-on-a-chip” devices, or protein arrays, or nanotechnology-based devices, or other emerging fields utilizing gold.

In another aspect, recombinant Streptavidin-GBP fusion is 5- to 10-fold more active in binding biotinylated molecules than is recombinant Streptavidin lacking the GBP domain when each are bound to gold.

In one embodiment, plasmid expression and protein production/purification of HiS₆-protein A-GBP, His₆-streptavidin-GBP, His₆-protein A-GBP-protein A, His₆-streptavidin-GBP-streptavidin, His₆-protein A-GBP-streptavidin, His₆-streptavidin-GBP-protein A, His₆-GBP, and His₆-GBP-GBP are disclosed.

In one aspect, there is no requirement to purify GBP or the desired protein prior to adsorbing them onto gold. The affinity and specificity of GBP to gold are sufficiently high, e.g., KD=1.5×10⁻¹⁰M to allow specific interaction in crude preparations containing many irrelevant proteins and other macromolecules.

The one to one relationship of GBP to fusion partner in the recombinant molecules enables the construction of uniform foundation layers containing high densities of functional protein. This can increase the sensitivity of detection in applications compared to that provided by conventional chemical attachment methods.

In a related aspect, the recombinant molecules can be constructed to orient recognition proteins appropriately to position their active sites outward from the gold surface to provide optimal interaction with target or substrate molecules. This is accomplished by placing the GBP domain at the N-, or C-termini, or within a surface loop of the recognition protein as indicated with linkers consisting of flexible amino acid sequences between domains. Conventional chemical attachments to GBP (Woodbury, et al., Sensors & Bioelectronics, 13:1117-1126, 1998) or other layers typically do not produce proper orientation to permit complete accessibility to binding sites on recognition proteins.

Expression plasmids disclosed herein can be readily adapted for the production of virtually any polypeptide. Once the expression hosts are created, unlimited quantities of many different GBP-containing recombinant proteins can be produced to create, for example, diverse arrays of proteins to facilitate proteomic research and drug screening. The gold-binding process is facilitated by the GBP domain common to each recombinant protein, thereby, ensuring attachment of all desired polypeptides, regardless of intrinsic, or lack of, attraction of the fusion partner to gold. Further, the one-to one relationship of GBP and its fusion partner allows the attachment to gold of equimolar amounts of hundreds or thousands of distinct recombinant molecules with different binding or enzyme activities. These benefits derived from the invention, herein, will significantly enhance the construction and performance of protein arrays, nanotechnology-based devices and the like.

The molecular approach described, herein, provides methods for introducing significant improvements in introducing a variety of functions to gold surfaces not possible by existing technology. For example, genetic engineering can produce a recombinant molecule containing GBP and the smallest possible form of a recognition protein that retains binding specificity. This provides at least three benefits. First, reduction of a protein to its specific binding domain eliminates other domains that may contribute complicating allosteric binding events or that could add to background interference. Second, in general, small functioning proteins are less susceptible than larger ones to proteolytic degradation when exposed to biologic fluids. Third, in the example of certain biosensing instruments, binding events occurring nearer the sensing surface produce stronger signals than those occurring farther away from the surface. Thus, the smaller the recognition protein, the higher the sensitivity of detection. A further benefit of the molecular approach is that appropriate modifications can be introduced into the protein sequence to produce a recombinant molecule with increased stability or other improvements. For example, if a region of the recombinant molecule is susceptible to proteolysis, introducing appropriate amino acid substitutions in the fusion protein may prevent degradation.

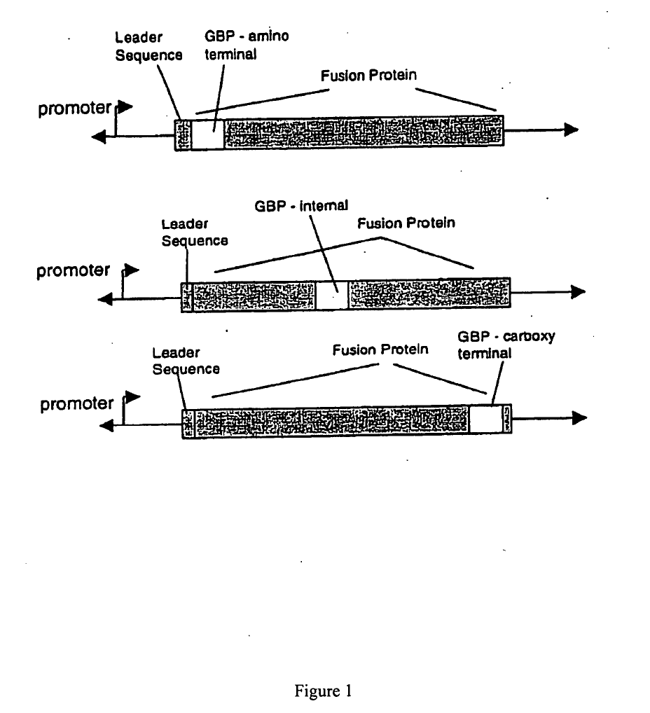

GBP fusion proteins can be arranged in several different ways as depicted in FIG. 1. The GBP sequence can be positioned at the amino terminus, internally or at the carboxyl terminus. The drawings represent the DNA sequence encoding the fusion protein portion of plasmid vectors that are expressed in bacterial, baculoviral, yeast, plant or mammalian cell hosts. It is apparent from the middle representation in FIG. 1 of an internally positioned GBP domain that two functional fusion partners, either identical partners or distinct partners can be placed in a single fusion protein. This feature will be described in detail in specific examples below.

In this disclosure, detailed methods for expressing GBP-based fusion proteins, rapid purification, characterization of activities, and specific examples for applications are described. Recognition proteins include, but are not limited to, protein A or G or related molecules, streptavidin or avidin or related molecules, single-chain antibodies, receptors, ligands, proteases, protease inhibitors, enzymes, enzyme inhibitors or any protein that specifically binds small molecules, cofactors or macromolecules. The latter group includes homo- or heterodimers or higher complexes of proteins and macromolecules required for a specific biologic function.

In one embodiment, the binding of the protein A- and streptavidin-GBP fusion proteins to colloidal gold under conditions is disclosed as recited, for example, in Examples 4 and 5. Optimization of binding includes modification of pH, salt concentration and other variables to establish preferred GBP binding conditions are disclosed. Protein A- or streptavidin-GBP binding and stability are measured using an enzymatic binding assay in which protein A and streptavidin is measured through it's ability to bind enzyme conjugated antibody.

In another embodiment, a lateral flow immunodetection system based on colloidal gold binding technology is disclosed. One of the key market applications for colloidal gold is as a detection reagent for immunodetection in lateral flow dipstick assays. Lateral flow tests are used for the specific qualitative or semi-quantitative detection of many analytes including antigens, antibodies, and even the products of nucleic acid amplification tests. One or several analytes can be detected simultaneously on the same strip. Urine, saliva, serum, plasma, or whole blood can be used as specimens. Extracts of patient exudates or fluids have also been successfully used.

The assay uses GBP-protein A bound to colloidal gold as a detection reagent. Samples contain or lacking IgG are placed on the absorption pad, and flow with the protein A conjugate. The presence of antibody in the test solution interferes with protein A binding to the IgG test strip, but develops a band at the anti-protein A control strip. In the absence of antibody protein A binds the IgG strip, and a band is visible. Streptavidin-GBP is used in similar fashion with biotinylated targets. For example, such particles can be used for detection of HIV gp120 or antibodies thereto.

A patent has been issued for a “Method of producing IGG-binding protein as fusion peptides and a vector therefor” that utilizes protein A (Lofdahl, et al., U.S. Pat. No. 5,100,788). Another patent has been issued for a process to produce fusion proteins containing streptavidin (Cantor et al., U.S. Pat. No. 4,839,293).

GBP-alkaline phosphatase chimera has been produced. The enzyme was fused to GBP solely as a reporter. Brown speculated that hybrid molecules containing metal-adhering peptides could bind to metallic sensor surfaces to provide more efficient procedures than are currently available. However, Brown does not disclose what these efficiencies are. Nor does Brown disclose how one reasonably skilled in the art can express and purify adequate amounts of stable hybrid molecules for commercial applications. The bacterial periplasmic expression system described by Brown produces only small quantities of GBP-alkaline phosphatase. Further, the expression of this particular fusion molecule may be preferentially favored because alkaline phosphatase is a normal periplasmic constituent. Many desired GBP-fusion proteins with commercial value may not be produced using Brown's expression system. Brown does not disclose alternative expression systems that those skilled in the art can use as a general strategy for the production of many different stable and active GBP-fusion proteins as described in the present invention. The prior art does not teach how stable GBP-fusion proteins can be expressed and purified in active form in large quantities as needed for commercial applications. Indeed, the prior art teaches that the expression and purification of each desired recombinant protein in active form are problematic. Brown does not disclose how those skilled in the art can overcome the unique set of difficulties encountered in the expression and purification of individual GBP-fusion proteins.

The present invention describes the fabrication of superior CG- or NG-polypeptide complexes compared to conventional methods. Bioactive polypeptides are fused to GBP to allow binding of polypeptides to CG, NG, or any type of gold-coated beads or particles.

In one embodiment, methods are disclosed for expressing and producing GBP-fusion proteins that contain bioactive polypeptides for the purpose of immobilizing the bioactivity on CG or NG. This technology has the potential of delivering any desired polypeptide directly to CG or NG regardless of the polypeptides intrinsic gold-binding capacity. It eliminates the use of inefficient or activity-destroying attachment methods and it provides reproducible stability. GBP optimally binds gold at pH 7 to 8, which is an ideal range for retention of bioactivity for most polypeptides. The 1:1 correspondence between the gold-binding and the bioactive polypeptide structures allows high-density surface binding. With optimum positioning of the GBP element, polypeptides can be tethered on surfaces to express full activity in the surrounding solution. In contrast, physical adsorption and chemical coupling methods can lead to surface denaturation and inactivation of polypeptides, and non-productive binding. The approach described herein provides high attachment efficiency, fidelity, and retention of activity that can lead to the development of more robust and sensitive forms of derivatized CG or NG.

Relatively few naturally occurring proteins bind strongly to CG or NG using standard procedures or retain full bioactivity when binding does occur. The presence of salt can prevent protein binding to gold. Many proteins are insoluble or bind other surfaces in low salt concentrations. Also, protein binding to CG or NG is favored at a pH close to the pI of the molecule. But many proteins of interest have reduced solubility near their pIs. Importantly, few small peptides of interest bind CG or NG directly and, therefore, many potential clinical and other testing applications are not possible using conventional methods. In a related aspect, the methods disclosed allow for gold binding of any fusion polypeptide to the GBP domain regardless of the intrinsic binding affinity of its partner and under conditions, i.e., pH 7 and moderate salt concentration that favor retention of activity and solubility of polypeptides. Further, the use of significantly less protein to saturate gold surfaces is observed because binding is facilitated and accelerated through GBP.

The methods and compositions disclosed allow for facile production of various iterations of CG and NG with GBP-fusion proteins containing bioactive polypeptides. Further, the invention allows for the use of small particles such as latex beads, plastic beads, or the like that have been coated with thin layers of gold to which GBP-fusion proteins containing bioactive polypeptides can be attached. The advantages of using gold-coated particles include, but are not limited to lower cost, more readily produced materials, easier to use materials, improved testing properties, greater stability during storage and testing, and wider application potential compared to existing methods.

The robust binding of GBP-fusion proteins to gold can lead to more significantly improved electron microscopy results. Sensitivity enhancement can be achieved because 100% of specific antibody or other capture molecule activity is expressed on NG. Use of NG-derivatized Protein A-GBP-HRP or Streptavidin-GBP-HRP also can increase sensitivity by developing HRP activity with substrates that form insoluble products. NG-derivatized with GBP-fusion proteins can be more durable during storage or use than existing reagents.

Protein A and related immunoglobulin-binding protein complexes with CG or NG are used extensively in IVD testing and research. Functional tests can be developed by binding analyte-specific antibody to protein A bound to CG or NG. Existing methods to prepare such complexes can be inadequate in supporting certain testing applications. For example, the lower limit of analyte detection in tests is generally 1 ng to 10 ng per mL of sample. This level of sensitivity is insufficient to detect many important clinical targets in biological fluids. Low testing sensitivity can occur because of random binding of protein A to CG or NG leading to less than full biological activity, low density binding of protein A, or instability of protein A-gold particle complexes during storage or testing. A new technology is needed to increase testing sensitivity.

Derivatizing CG or NG with GBP-Protein A or the like can significantly increase testing sensitivity and allow additional new testing applications. Sensitivity enhancement occurs because gold binding is restricted to the GBP, thereby, maximizing the density of surface bound molecules with full expression of protein A activity. Also, GBP binding to gold is essential irreversible unlike physically adsorbed protein A. Thus, protein A activity is not lost during storage or testing.

In one embodiment, the binding of GBP-Streptavidin or related fusion proteins to CG or NG is disclosed. Most polypeptides can be biotinylated and then attached to GBP-streptavidin bound to CG or NG to enable a specific testing application.

In another embodiment, a method to introduce two distinct bioactivities onto CG or NG is disclosed. In this method, protein A or the like can be attached to one terminus of GBP in a recombinant fusion protein. To the other terminus of GBP is attached another polypeptide, e.g.,—but not limited to—the enzyme horse radish peroxidase (HRP). The entire fusion protein is configured as Protein A-GBP-HRP or HRP-GBP-Protein A.

These bi-functional molecules can be useful in IVD testing. Diagnostic applications using lateral flow strip technology based on CG rely on the development of color arising from the concentration of antibody-derivatized colloidal gold (typically red/brown shades) at appropriate locations on the test strip. Some tests are designed so that the lack of color development is the readout. Test results are semi-quantitative, and the lower detection level is approximately 1 ng of target molecule/mL of sample. This level of sensitivity is adequate for many clinical tests, but insufficient for others. Readouts approaching the lower limit of detection testing can be unreliable. The present invention describes a method to significantly increase the sensitivity of lateral flow testing using HRP-GBP-Protein A bound to CG. Testing specificity can be conferred by binding specific antibody to the Protein A domain. In convention lateral flow testing, a positive readout due to the color of concentrated colloidal gold increases rapidly to a certain level, and may diminish over time. In contrast, the presence of HRP in the fusion protein as disclosed on the same amount of CG used in conventional testing can provide significantly enhanced test sensitivity as a result of signal amplification possible through enzymatic catalysis over time. HRP substrates are available that can be converted to insoluble, colored products in the vicinity of concentrated HRP. HRP-GBP-Protein A provides a versatile testing system that can be used conventionally where colloidal gold color development is the primary readout, or by developing HRP activity in instances of low sensitivity or uncertain test results. A positive test on a lateral flow strip can result in a precipitin band where HRP is concentrated when the test strip is dipped into a solution containing substrate and hydrogen peroxide. Alternatively, very sensitive competition binding assays can be designed whereby a positive readout is the absence of a precipitin band.

In another embodiment, we describe a method to introduce two distinct bioactivities onto CG or NG whereby streptavidin, or avidin or the like can be attached to one terminus of GBP in a recombinant fusion protein. To the other terminus of GBP is attached another polypeptide, e.g.,—but not limited to—the enzyme horseradish peroxidase (HRP). The entire fusion protein is configured as Streptavidin-GBP-HRP or HRP-GBP-Streptavidin.

In a manner similar to that described above for Protein A-GBP-HRP, Streptavidin-GBP-HRP can be used to support IVD testing with enhanced sensitivity. For example, any specific antibody regardless of class or any active antibody fragment can be biotinylated and then bound to CG derivatized with Streptavidin-GBP-HRP. Tests can be developed as described above.

Streptavidin-GBP-HRP and Streptavidin-GBP are unique molecules that allow biotinyl-peptide antigens to be readily introduced onto CG or NG. Those skilled in the art can use each fusion protein to support IVD testing when specific antibody is also used. Streptavidin-GBP-HRP has the additional advantage of enhancing sensitivity through enzymatic activity as described above.

In another embodiment, a method is described to introduce two distinct bioactivities onto CG or NG whereby any polypeptide with biological activity, e.g.,—but not limited to—peptide antigens, can be attached to one terminus of GBP in a recombinant fusion protein. To the other terminus of GBP is attached another polypeptide, e.g.,—but not limited to—the enzyme horseradish peroxidase (HRP). The entire fusion protein is configured as Antigen-GBP-HRP or HRP-GBP-Antigen.

Those skilled in the art can develop assays with these fusion proteins by also using specific antibody recognizing the antigens. Examples of specific tests made possible by the present invention include, but are not limited to, HIV detection when the antigen is gp120 or related polypeptide; prostate cancer diagnosis when the antigen is prostate specific antigen or related polypeptide; detection of a variety of autoimmune diseases when specific antigens and related polypeptides are utilized; detection of innumerable infectious agents when appropriate specific antigens or related polypeptides are utilized; and detection of various bioterrorist agents when specific antigens or related polypeptides are used.

Novel GBP fusion proteins can be used to establish significantly improved lateral flow strip immunodetection systems based on CG or NG binding technology. Lateral flow strip tests are used for qualitative or semi-quantitative detection of many analytes including antigens, antibodies, and the products of nucleic acid amplification. One or several analytes can be detected simultaneously on the same strip. Urine, saliva, serum, plasma, or whole blood can be used as specimens.

In one embodiment, assays use GBP-protein A bound to CG or NG as a detection reagent. Samples containing or lacking IgG are placed on the absorption pad, and flow with the protein A conjugate. The presence of antibody in the test solution interferes with protein A binding to the IgG test strip, but develops a band at the anti-protein A control strip. In the absence of antibody protein A binds the IgG strip, and a band is visible.

In another embodiment, streptavidin-GBP is bound to CG or NG and the complex is used in similar fashion described above after attaching biotinylated molecules that enable testing.

In another embodiment, Protein A-GBP-HRP is bound to CG or NG and the complex is used in similar fashion described above in lateral flow strip assays. Developing HRP activity with appropriate substrates that result in insoluble products can increase test sensitivity.

In another embodiment, Streptavidin-GBP-HRP is bound to CG or NG and the complex is used in similar fashion described above in lateral flow strip assays. Developing HRP activity with appropriate substrates that result in insoluble products can increase test sensitivity.

In certain applications the chemical, physical, or surface charge properties of CG or NG preclude their effective use in IVD testing. Small spherical beads composed of non-gold materials have been used as substitutes for CG or NG to support lateral flow strip testing and other applications. For certain applications it can be desirable to combine the properties of non-gold beads and the properties conferred by a surface comprised of a thin layer of pure gold. The present invention provides a method for adding thin layers of pure gold to the surface of non-gold materials to allow surface binding of GBP-fusion proteins. For certain materials it may be necessary to coat the surface first with a thin layer of chromium prior to the addition of gold. The inventive concept integrates the robust surface chemistry of GBP-fusion proteins that bind to a thin layer of surface gold with certain superior properties of a various non-gold core materials to significantly increase the number of potential applications.

While the inventive concept uses the field of IVD testing as an example, its scope is not limited to IVD testing. For example, in another embodiment, non-gold biomaterials can be coated with thin layers of gold to allow robust surface binding of GBP-fusion proteins that are also biocompatible.

In another aspect, medical devices comprised of non-gold materials can be coated with a thin layer of gold without altering the basic electrical, physical, or mechanical properties of the substrate material. GBP-fusion proteins can then be added to the surface to provide biological activity or a biocompatible film or protective barrier.

In another aspect, micro-array chips and other devices comprised of non-gold materials can be coated with a thin layer of gold without altering the basic chemical, electrical, or physical properties of the underlying substrate material. GBP-fusion proteins can then be added to the surface to provide biological activity.

In another aspect, bioimaging or biocontrast agents comprised of non-gold materials can benefit using GBP-fusion proteins by coating the agents with a thin layer of gold.

In another aspect, therapeutic materials including, but not limited to, radioactive or other cytotoxic metals or other cytotoxic materials can be coated with a thin bioprotective layer of gold; derivatized with GBP-fusion proteins containing specific antibodies, or cell receptor ligands, or other cell specific binding molecule, or other tissue specific binding molecule; and the derivatized material can be targeted and concentrated on or in specific cells, tissues, or organs, or cancerous tumors.

In one aspect, a fusion protein consisting of GBP and any of a variety of tissue collagens can be bound to a biosensing device to measure collagenase activity in tissue extracts, or cell extracts, or body fluids, or cell culture medium.

In another aspect, a fusion protein consisting of GBP and tissue elastin can be bound to a biosensing device to measure elastase activity in tissue extracts, or cell extracts, or body fluids, or cell culture medium.

In another aspect, a fusion protein consisting of GBP and fibrin can be bound to a biosensing device to measure fibrinolytic activity in tissue extracts, or cell extracts, or body fluids, or cell culture medium.

In another aspect, a fusion protein consisting of GBP and any of a variety of blood coagulation factors can be bound to a biosensing device to measure the specific activity of factor activation in tissue extracts, or cell extracts, or body fluids, or cell culture medium.

In another aspect, a fusion protein consisting of GBP and any of a variety of blood complement proteins can be bound to a biosensing device to measure the specific activity of protein activation in tissue extracts, or cell extracts, or body fluids, or cell culture medium.

In another aspect, a fusion protein consisting of GBP and any of a variety of proteins involved in the process of apoptosis can be bound to a biosensing device to measure the specific protein activation activity in cell extracts or cell culture medium.

In another aspect, a fusion protein consisting of GBP and a specific polypeptide substrate of a protease on or secreted from cells can be bound to a biosensing device to measure the specific protease activity on cells, or in cell extracts, or secreted by cells into culture medium or body fluids.

In another aspect, a fusion protein consisting of GBP and a specific polypeptide substrate of a protease required for viral processing can be bound to a biosensing device to measure the specific protease activity in tissue extracts, or cell extracts, or body fluids, or in cell culture medium.

In another aspect, a fusion protein consisting of GBP and a specific polypeptide substrate of a protease secreted from or residing on a parasite can be bound to a biosensing device to measure the specific protease activity in tissue extracts, or cell extracts or body fluids, or in cell culture medium.

In many other aspects, a fusion protein consisting of GBP and a specific polypeptide inhibitor(s) of a protease can be bound to a biosensing device to detect the presence of a protease in test samples. The device can be used to quantify protease levels in tissue extracts, plant extracts, parasite extracts, cell extracts, body fluids, or in cell culture medium.

Woodbury and coworkers (Woodbury, et al., Sensors & Bioelectronics, 13:1117-1126, 1998; and U.S. Pat. No. 6,239,255) disclose a method for the chemical attachment of molecules to a GBP foundation layer on gold. Their methods are limited to the construction of biosensing instruments based on the optical principle of surface plasmon resonance. No disclosures or claims are made for the expression, purification, and applications of recombinant GBP-fusion proteins as conceived in the present invention.

In one embodiment, methods for constructing unique expression vectors, for the production of large quantities of stable fusion proteins, for the determination of the activities of all fusion partners, and specific commercial applications for GBP-fusion proteins are disclosed. In a related aspect, general expression and purification procedures capable of producing large quantities of stable, active fusion proteins with little effort and cost, thereby increasing the prospect of developing commercial applications are disclosed.

The following examples are intended to illustrate but not limit the invention.

### EXAMPLE 1

### Plasmid Design for Expression of GBP Fusion Proteins

Recombinant fusion proteins are produced by expression of plasmid constructs encoding the protein of interest fused with the GBP. The plasmid constructs include a selectable marker including but not limited to ampicillin resistance, kanamycin resistance, neomycin resistance or other selectable markers. Transcription of the GBP fusion protein is driven by a regulatable promoter specific for expression in bacteria, yeast, insect cells or mammalian cells. The construct includes a leader sequence for expression in the periplasmic space, for secretion in the media, or for expression in inclusion bodies in bacterial cells, or for secretion in yeast or mammalian cells. Plasmid constructs include multiple cloning sites for insertion of protein sequences in frame with respect to the GBP polypeptide. The GBP sequence can be inserted at the amino-terminal or C-terminal end of fusion partners or inserted within the coding sequence of the fusion partner. More than one GBP domain can be fused to a single fusion partner. More than one fusion partner can be fused to a single GBP sequence.

Herein described is the design of a modular set of vectors to support the production of amino and carboxyl terminal fusion proteins in E. coli expression systems. Included are the addition of amino or carboxy affinity tags for purification; the addition of flexible linking sequences between domains to provide independent activity of fusion partners; the presence of a specific cleavage site to disconnect fusion partners if desired; and the requirement for highly regulated expression where toxicity of the over-expressed fusion protein could limit production.

General Methods:

Media. Strains and Transformation: LB media (Bacto L B broth, Miller, from Difco) was used as the basic growth media throughout the course of this study. The antibiotic ampicillin was used at a concentration of 150 μg/ml on plates and at 100 μg/ml in liquid media for the selection and growth of plasmid containing cells. NovaBlue cells from Novagen served as the E. coli host for transformation and expression. Transformations were performed according to the manufacturer's protocol.

Molecular Biology Supplies: All restriction endonucleases and T4 DNA ligase were purchased from New England Biolabs and the kit for DNA sequencing for the Big Dye terminator cycle sequencing from PE/ABI. Plasmid DNAs were made using the miniprep plasmid kits from Qiagen and DNA was extracted from agarose gel slices with is gel extraction kits from either Qiagen or Eppendorf. All reagents were used according to the manufactures' protocols.

Construction of the expression plasmid for Protein A-GBP fusion protein.

The plasmid pSB3053 obtained from S. Brown (Brown, Nat. Biotechnol. 15:269-272, 1997) was used as the source of the GBP fragment containing seven repeats of the peptide MHGKTQATSGTIQS (SEQ ID NO:17). Upon DNA sequencing it was found that the last repeat carried a substitution of the threonine residue in the fifth position for an isoleucine. All the fusion proteins constructed in this work have this substitution.

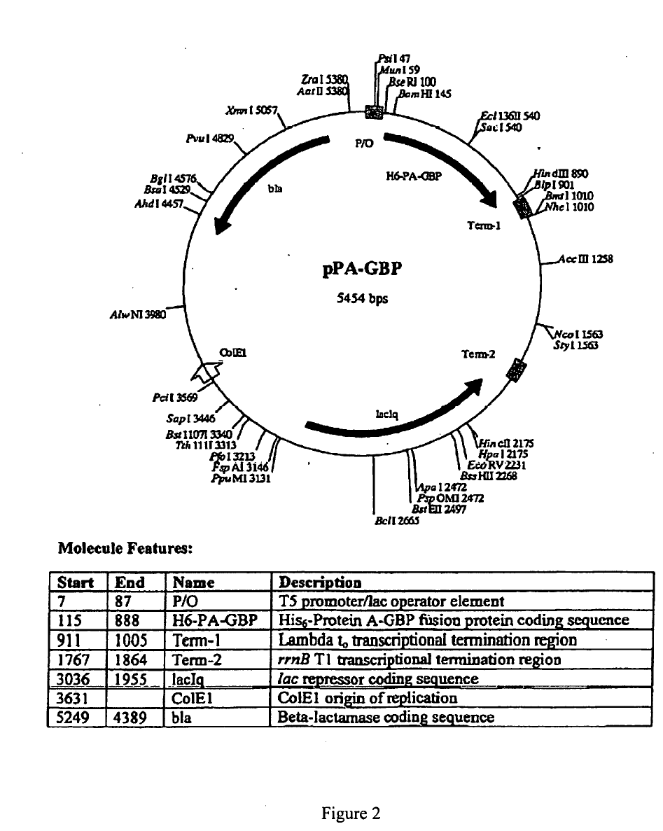

An EcoR I-Xho I fragment encompassing the GBP coding sequence was excised from pSB3053 and adapted at the 3′ end to include coding triplets for the amino acids EGP and a stop codon. Oligonucleotides BH3 (5′ TCG AGG GTC CGT AAT A 3′: SEQ ID NO:18) and BH4 (5′ AGC TTA TTA CGG ACC C 3′: SEQ ID NO:19) were annealed to obtain an adaptor with Xho I and Hind III cohesive ends. The EcoRI-Xho I GBP containing fragment and the adaptor were assembled in pUC 18 and cut with EcoR I and Hind III in a three-part ligation to obtain plasmid pBHI-1. The Bsl I-Hind III fragment from pBHI-1 carrying the GBP coding sequence was adapted at its 5′ end to include an in-frame linker sequence with an Asn-Gly hydroxylamine sensitive cleavage site. Oligonucleotides BH1 (5′ CTG GTA GTG GCA ATG GTC ATA TGC 3′: SEQ ID NO:20) and BH2 (5′ TAT GAC CAT TGC CAC TAC CAG AGC T 3′: SEQ ID NO:21) were annealed to obtain an adaptor with Sac I and Bsl I cohesive ends. The adaptor also incorporates an Nde I site at the methionine codon of the first GBP repeat for ease of adaptation of the GBP fragment with any desired in-frame sequence. Plasmid pBHI-2 was generated with the Bsl I GBP fragment this adaptor and pUC19 linearized with Sac I and Hind III, in a three-part ligation. The nucleotide sequence of the Sac I-Hind III, double-adapted GBP fragment was confirmed by DNA sequencing. The Sac I-Hind III fragment from pBHI-2 was cloned between the Sac I and Hind III sites of PEZZ18 (Amersham) for an in-frame fusion with the two Z domains of staphylococcal Protein A (Nilsson, et al., Protein Eng 1: 107-113, 1987) to obtain plasmid pBHI-3. The final expression plasmid for the cytoplasmic production of the His-tagged fusion protein was constructed by ligating the Protein A-GBP containing Fsp I-Hind III fragment from pBHI-3 and a short adaptor sequence formed by oligonucleotides BHI I and BH12 (5′ GAT CCG GTT CTG GTG C 3′ (SEQ ID NO:22) and 5′ GCA CCA GAA CCG 3′ (SEQ ID NO:23), respectively) into pQE-80L (Qiagen, Inc) cut with BamH I and Hind III. The resulting plasmid, called pPA-GBP, is depicted in FIG. 2. The nucleotide sequence of the encoded fusion protein was confirmed by DNA sequencing. The complete DNA sequence of pPA-GBP and the amino acid sequence of the fusion protein appear in the Sequence Listing section at the end of this document.

Construction of the expression plasmid for Streptavidin-GBP fusion protein. The coding sequence for core streptavidin residues 13-139 of the mature polypeptide (Sano, et al., J Biol Chem 270:28204-28209, 1995) was derived from a pUC18-based plasmid obtained from Dr. P. Stayton (Chilkoti et al., Proc Natl Acad Sci USA 92:1754-1758, 1995). A Sac I restriction site was engineered into the coding sequence to allow fusions to the shortened version of streptavidin, residues 13-133 (Sano, et al., J Biol Chem 270:28204-28209, 1995). For this, an EcoR 1-Mlu I fragment encoding the partial core streptavidin sequence was linked to an adaptor with Mlu I and Hind III cohesive ends (formed using oligo pairs BH7/BH8, 5′ CGC GTG GAA ATC CAC CCT GGT TGG TCA 3′ (SEQ ID NO:24)/5′ GTG TCG TGA CCA ACC AGG GTG GAT TTC CA 3′ (SEQ ID NO:25) and BH9/BH10 5′ CGA CAC CTT CAC CAA AGT TTC GAG CTC 3′ (SEQ ID NO:26)/5′ AGC TTG AGC TCG AAA CTT TGG TGA AG 3′ (SEQ ID NO:27)) and inserted into pUC18 cut with EcoR I and Hind III to yield pBHI-5. The nucleotide sequence of the total EcoR I-Hind III insert in pBHI-5 was confirmed by DNA sequencing.

Using an Nde I site present at the initiating methionine of the adapted core streptavidin sequence in pBHI-5, the Nde I-Hind III fragment encoding core streptavidin was cloned into the expression vector pQE-80L (Qiagen, Inc), digested with BamH I and Hind III. A short adaptor sequence with BamH I and Nde I cohesive ends, formed with the oligo pair BH17/BH18 (5′ GAT CCG GTT CTG GTG GCC A 3′ (SEQ ID NO:28)/5′ TAT GGC CAC CAG AAC CG 3′ (SEQ ID NO:29)) was used for linking.

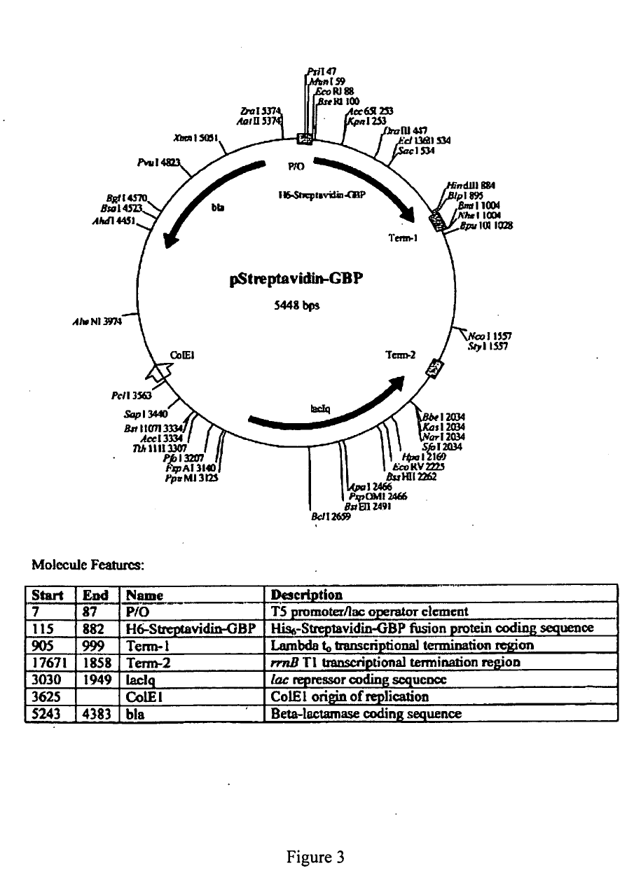

The resulting plasmid called pBHI-7 can produce a N-terminal His-tagged core streptavidin molecule residues 13-133, ending with the added amino acid residues SSSSILS (SEQ ID NO:30). To express the His-tagged core streptavidin-GBP fusion protein, the engineered Sac I site in the core streptavidin sequence (see above) was utilized to link the Sac I-Hind III GBP encoding fragment from pBHI-2 to generate the expression plasmid pStreptavidin-GBP which has the basic backbone of the expression vector pQE 80L (Qiagen, Inc). The plasmid map, pStreptavidin-GBP is depicted in FIG. 3 and relevant DNA and amino acid sequences appear in the Sequence Listing section at the end of this document.

In summary, vectors are disclosed for the expression of His₆-protein A-GBP, His₆-streptavidin-GBP and His₆-streptavidin lacking the GBP as a control protein. In addition GBP was subcloned as a modular cassette to support the development of future recombinant fusion proteins.

The expression constructs contain DNA that encodes repeating glycyl-seryl sequences to provide flexible linkers between domains for maximizing independent activities of domains.

The expression constructs contain DNA that encodes specific chemical cleavage sites including, but not limited to, asparaginyl-glycyl or aspartyl-prolyl bonds (Bornstein and Balian, Methods Enzymol 47:132-145, 1977; Szoka, et al., DNA 5:11-20, 1986). The invention also provides for DNA that encodes specific protease cleavage sequences for Factor Xa or Enterokinase and the like (Jenny, et al., Protein Expr Purif 31:1-11, 2003; Wang, et al., Biol Chem Hoppe Seyler 376:681-684, 1995).

The expression constructs contain DNA that encodes an affinity “tag” sequence, for example, but not limited to, polyhistidine, V-5 epitope, or FLAG epitope to facilitate rapid, one-step purification of fusion proteins (Dobeli, et al., U.S. Pat. No. 5,047,513; Chen, et al., Eur J Biochem 214:845-852, 1993; Terpe, Appl Microbiol Biotechnol 60:523-533, 2003).

### EXAMPLE 2

### Expression of GBP-Fusion Proteins

The GBP-fusion constructs for all examples were transfected into NovaBlue cells (Novagen). For expression, an overnight culture of the transformants grown in LB broth+ampicillin at 37° C. was diluted into fresh media and grown with vigorous shaking till the OD measured at 600 nm was between of 0.3-0.4. Isopropyl .beta.-D-thio-galactopyranoside was added to a final concentration of 4 mM and the incubation was continued for another 4 hours. The cells were collected by centrifugation, washed once with 150 mM KCl and frozen.

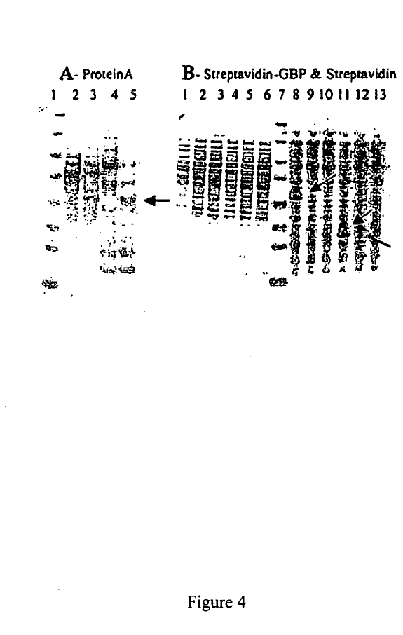

In preliminary experiments, induced and non-induced cells were first extracted in B-Per (Pierce), a gentle buffer for lysis of bacteria to recover soluble proteins. The extract was centrifuged to clarify the solution and the pellet was extracted directly in SDS-PAGE sample buffer to recover insoluble proteins. All samples were analyzed by SDS-PAGE and staining with a colloidal form of coomasie blue (Invitrogen). The results of these experiments shown in FIG. 4 indicate that high levels of His₆-protein A-GBP and His₆-streptavidin-GBP fusion proteins were produced by induced cells and little, if any, protein was observed in non-induced cells. Thus, the repressible/inducible system functioned as expected. Further, there was no apparent proteolytic degradation of the fusion protein during culture or the extraction procedure. In the case of His₆-protein A-GBP, some of the fusion protein appeared to be in the soluble fraction, but most was observed in the SDS-PAGE sample buffer extracts. In contrast, essentially all of the His₆-streptavidin-GBP fusion was insoluble and required SDS to extract the protein. A His₆-streptavidin construct lacking the GBP domain was also expressed and the resulting protein had solubility properties similar to those of the molecule containing GBP.

The fusion partners were observed to bind gold powder directly from the crude cellular extracts as evident by SDS-PAGE analysis of the gold powder. A few, very abundant E. coli proteins also bound gold but it was clear the GBP-fusions preferentially bound gold.

### EXAMPLE 3

### Purification of GBP-Fusion Proteins

Larger cultures were grown to produce sufficient fusion proteins for purification and characterization. To extract proteins under “native” conditions for subsequent purification, the bacteria were resuspended in 50 mM sodium phosphate buffer, pH 8.0, containing 0.5M sodium chloride and 10 mM imidazole to a final density approximately 20 times greater than that of the original cultures. Cells on ice were lysed by sonication at medium power and interval setting of 50% to give an intermittent pulse for 30 seconds. This was repeated for 6 cycles with one-minute rest on ice between cycles. Following each cycle, the optical density at 600 nm was recorded to assess cell lyses. The sonicated suspension was centrifuged 5,000×g for 10 min to remove cell debris and insoluble proteins from the soluble fraction. The resulting pellet was extracted in a “denaturing” solution of 20 mM sodium phosphate buffer, pH 7.8, containing 6M guanidine HCl (Gu-HCl) and 0.5M sodium chloride and the suspension was centrifuged to remove insoluble material.

In the case of the streptavidin fusion proteins, the cells were extracted only with 20 mM sodium phosphate buffer, pH7.8, containing 6M Gu-HCl and 0.5M sodium chloride.

Purification of His₆-protein A-GBP, His₆-streptavidin-GBP-, and His₆-streptavidin fusion proteins.

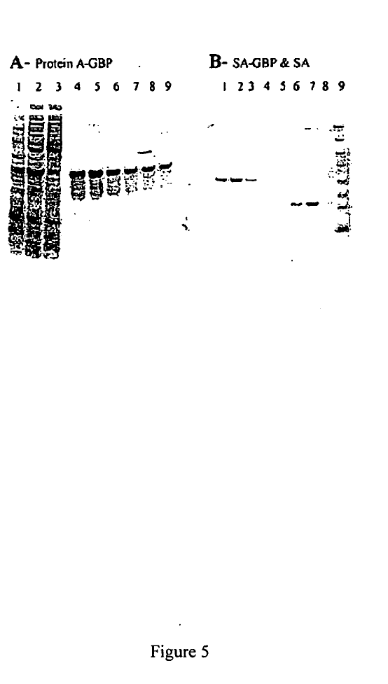

The His₆-tag recombinant proteins, were purified on ProBond nickel-resin columns (Invitrogen) as recommended by the manufacturer. Material in the two extracts, i.e., under native conditions for soluble proteins or denaturing conditions for insoluble proteins, was incubated with individual Probond Nickel resin columns, washed, and eluted as recommended by the manufacturer. Analysis by SDS-PAGE shown in FIG. 5 indicated that the final preparations were 90%-95% pure accompanied by proteolysis of a small amount of material, probably at the GBP domain. Initial extracts did not include protease inhibitors, but future preparations will include PMSF and a commercial “cocktail” of protease inhibitors. The optical density at 280 nm of the eluate fractions was recorded and the peak fractions from each column were pooled, aliquoted and stored at −20° C. Interestingly, sonication solubilized at least 80% of the total His₆-protein A-GBP. Thus, one-step purification of stable recombinant His6-protein A-GBP, His₆-streptavidin-GBP, and His₆-streptavidin proteins was possible in just a few hours from cell extraction to pure protein.

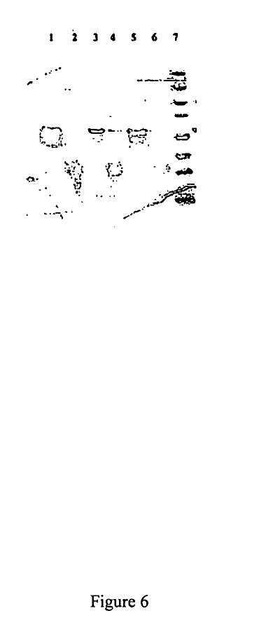

The inclusion of an Asn-Gly bond, susceptible to hydrolysis in 2M hydroxylamine and 4M urea at pH 9.5, allowed for physically dissociation of GBP from protein A as shown in FIG. 6. As a method to achieve limited digestion of proteins, urea is required to unfold proteins to make any Asn-Gly bonds fully accessible to hydroxylamine. However, because of the exposed location of our inserted Asn-Gly bond efficient hydrolysis was achieved without adding urea in just a few hours. Further, it was possible to hydrolyze the fusion protein while it was bound to gold powder. Thus, selectively hydrolyze fusion proteins is possible at the inserted Asn-Gly site even when fusion partners contain such bonds, especially if even less stringent conditions can be employed.

### EXAMPLE 4

### Characterization of GBP-Fusion Proteins

Colorimetric assays were developed to determine gold-binding activity of GBP and fusion partner activities of the purified recombinant proteins. Spherical gold powder (Sigma-Aldrich), 1.5 to 3 micron in size, was washed overnight at room temperature in hydrofluoric acid to remove contaminants (Brown, Nat. Biotechnol. 15:269-272, 1997). Samples containing 0 to 330 picomole of purified His₆-GBP-protein A or native protein A (Sigma) were diluted in 1 mL 10 mM potassium phosphate, pH 7.0, containing 100 mM potassium chloride and 1% triton X-100 (PKT buffer) and incubated in 2 mL centrifuge tubes with 1 mg of gold powder for 5 min at room temperature with gentle mixing. Samples containing 0 to 22 picomole of purified recombinant His₆-streptavidin-GBP or His₆-streptavidin were similarly prepared. Gold powder was collected by centrifugation at 10,000×g for 1 min and incubated in 1 mL of phosphate buffered saline (PBS), pH 7.4, containing 2 mg bovine serum albumin (BSA)/mL for 5 min with mixing. The gold powder was then rinsed twice in a 1:1 solution of PKT and PBS/BSA buffers.

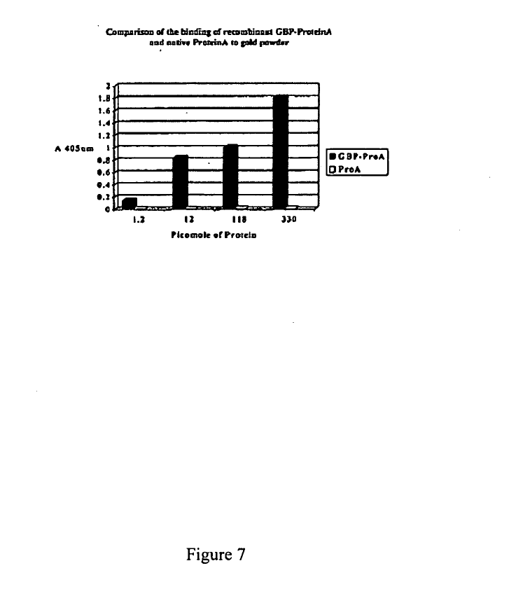

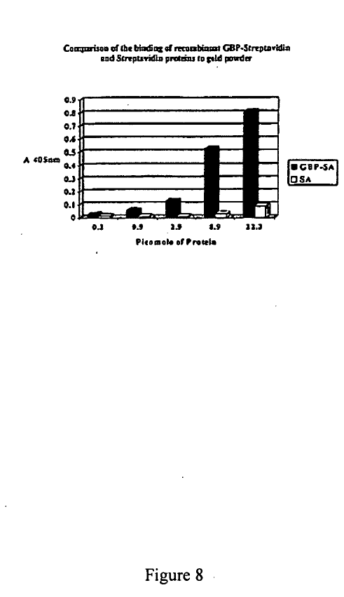

In the case of the protein A samples, mouse monoclonal IgG1 antibody (anti-FLAG, Sigma-Aldrich) labeled with alkaline phosphatase was incubated at room temperature at a dilution of 1:1000 with the gold powder in 1 mL of a 1:1 solution of PKT and PBS/BSA buffers for 15 min with mixing. To assess streptavidin activity, biotinylated goat antiserum with specificity to mouse immunoglobulin (Sigma-Aldrich) was incubated at room temperature at a dilution of 1:1000 with the gold powder in 1 mL of a 1:1 solution of PKT and PBS/BSA buffers for 15 min with mixing. The gold powder was rinsed twice in 1 mL of 1:1 solution of PKT and PBS/BSA buffer, and incubated with 1 mL of a 1:1000 dilution of mouse monoclonal (anti-rabbit) conjugated alkaline phosphatase in a 1:1 solution of PKT and PBS/BSA buffers for 15 min with mixing. The gold powder was washed twice in 1 mL of 1:1 solution of PKT and PBS/BSA buffer, transferred to unused centrifuge tubes, and assayed for alkaline phosphatase activity in 1 mL of p-nitrophenylphosphate in 50 mM Tris-HCl, pH 8.0, (51 mg in 25 mL) at room temperature with mixing over time. The reaction was stopped by removing the gold by centrifugation. The optical densities at 405 nm of the supernatant fluids were recorded. The results shown in FIGS. 7 and 8 indicate that the recombinant proteins contain both functional GBP domain and fusion partner activities. Further, the results establish the remarkable ability of GBP to facilitate specific gold binding of proteins at very low concentrations compared to direct adsorption of protein A and His₆-streptavidin which, lacking the GBP domain, bind minimally to gold powder in PKT buffer.

The concentration range for the recombinant streptavidin proteins was less than that for protein A because these proteins were still in 6M Gu-HCl following purification and preliminary studies indicated that gold binding by His₆-streptavidin-GBP was inhibited at relatively low Gu-HCl concentrations. This was not unexpected because GBP contains no disulfide bonds to help stabilize the polypeptide's tertiary structure. Future studies will be performed in the absence of Gu-HCl to determine levels of protein needed to saturate gold, however, the observation of the effect of this agent on gold-binding was fortuitous. Additional studies were conducted to gain further insight regarding GBP gold binding properties. There is a possibility that inhibition of gold binding was not a direct effect of Gu-HCl on GBP, but rather the guanidinium ion could compete with GBP for binding sites on gold in PKT buffer. If so, the ions must bind tightly to gold to block GBP attachment. Therefore, samples of gold powder were washed with up to 0.5M Gu-HCl in PKT buffer, recovered by centrifugation prior to binding His₆-streptavidin-GBP in PKT buffer, and compared to the binding of fusion protein to gold not washed with Gu-HCl. The results indicated near identical His₆-streptavidin-GBP binding to gold powder whether or not the powder was pre-washed with 0.5M Gu-HCl suggesting that the original observation of Gu-HCl inhibition of fusion protein binding to gold was a direct effect of the agent on GBP.

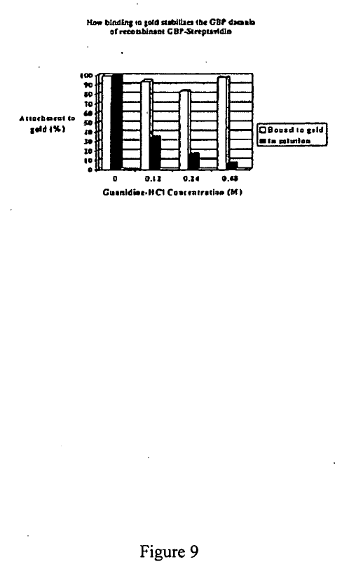

This study was followed by one to assess the stability of His₆-streptavidin-GBP already attached to gold powder in the presence of PKT buffer containing increasing concentration of Gu-HCl. The results shown in FIG. 9 indicate that once formed the GBP/gold interaction is remarkably stable when exposed to a strong chaotropic agent such as Gu-HCl. Indeed, following incubation in 3M and 6M Gu-HCl, there was 70% and 30% retention of His₆-streptavidin-GBP binding to gold powder, respectively. The observed stability for the GBP/gold interaction in this study is likely an underestimate since hydrofluoric acid-treated gold powder still contains contaminants that may preclude optimum interaction of some molecules of GBP with gold (Brown, Nat. Biotechnol. 15:269-272, 1997). Nevertheless, the results indicate that robust biosensors and other applications will be supported by these GBP-fusion proteins.

### EXAMPLE 5

### Construction and Characterization of Biosensors

Surface plasmon resonance (SPR)—an optical principle-biosensors were constructed on a fully integrated miniature SPR transducer, called Spreeta, from Texas Instruments (Melendez, et al., Sensors & Actuators B, 35, 36:212-216, 1996). Sensor chips were coated with recombinant His₆-protein A-GBP and His₆-streptavidin-GBP and the performance of each was compared to that of control sensors constructed with native protein A or recombinant streptavidin lacking the GBP domain. Solutions were delivered by a peristaltic pump at a flow rate of 0.2 mL/min at room temperature through a flow cell attached to each sensor. Clean sensing surfaces were rinsed initially for 10 min in 10 mM potassium phosphate buffer, pH 7.0 containing 10 mM potassium chloride and 1% Triton X-100 (PKT buffer) followed by solutions of PKT buffer containing test proteins. In the case of protein A-GBP or native protein A, the gold sensing surfaces were incubated for 10 min with 12 picomole of protein/mL For recombinant His₆-streptavidin-GBP or His₆-streptavidin 4.5 picomole of each/mL was used. Again, the presence of Gu-HCl precluded using higher amounts of protein. Future studies will use solutions without Gu-HCl, but in the current studies the concentration of proteins was sufficient to saturate the tiny sensing area. Following the application of protein, the sensors were rinsed with PKT buffer and then phosphate buffered saline, pH 7.4, containing 2 mg bovine serum albumin/mL (PBS/BSA buffer) for 10 minutes each. This completed the process to construct a sensor.

All antibodies were diluted at 1:1000 in PBS/BSA buffer for sensing evaluation. All solutions flowed over the sensing surface for 10 min each with the exception of 20 min for 0.1 M glycine-HCl, pH 2.0, used to regenerate the surface. Refractive index (RI) vs. time was recorded by Spreeta software on a laptop commuter.

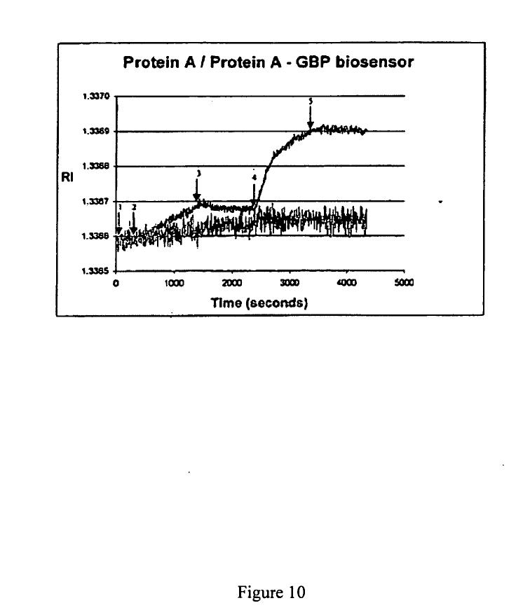

a) Recombinant His₆-protein A-GBP and native protein A. To evaluate their performance each sensor was exposed to mouse monoclonal IgG (anti-FLAG), rinsed in PBS/BSA buffer, exposed to polyclonal goat anti-mouse, and rinsed in PBS/BSA buffer. This procedure effectively eliminates non-specific antibody binding. The results shown in FIG. 10 indicate excellent gold- and immunoglobulin-binding activities for His₆-protein A-GBP as anticipated from the results of studies with gold powder. Also, as expected, there was no evidence of binding with native protein A. Exposure of the His₆-protein A-GBP based sensor to 0.1M glycine-HCl, pH 2.0, regenerated the sensing surface and allowed a second high-quality analysis. No evidence of sensing fouling in the presence of BSA or antibodies was observed.

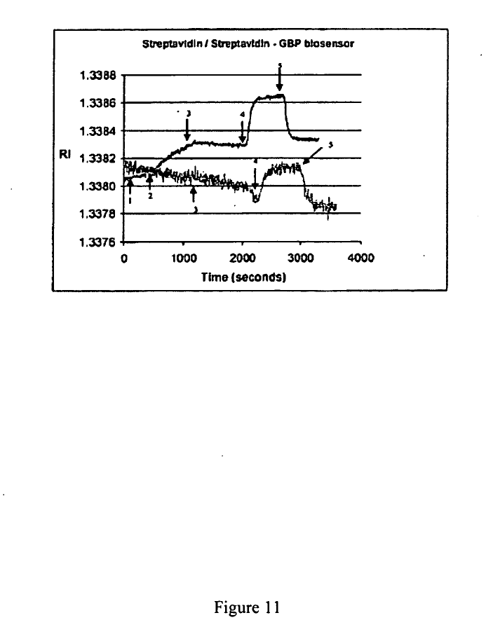

b) Recombinant His₆-streptavidin-GBP and His₆-streptavidin. Each sensor was exposed to biotinylated goat anti-mouse antibody, rinsed in PBS/BSA, buffer, exposed to mouse IgG (conjugated with alkaline phosphatase), and rinsed in PBS/BSA buffer. The results shown in FIG. 11 indicate that a very robust sensor was constructed with His₆-streptavidin-GBP, but not with His₆-streptavidin lacking GBP. The rapid increase in RI when mouse IgG was introduced was due to glycerol in the stock preparation. The signal for capturing mouse IgG by anti-mouse antibody held firm to His₆-streptavidin-GBP was significant, but less than expected probably because some of the epitopes on the conjugated target were blocked. As with a) above the sensor was regenerated by removing the mouse IgG in 0.1M glycine-HCl, pH 2.0, allowing a second analysis for capture of mouse IgG. There was no evidence of sensor fouling by BSA or antibodies.

The sensor constructed with His₆-streptavidin without GBP completely lacked activity. While the protein was applied to the sensor it was evident that material bound initially to the sensing surface, but was partially washed off during the extensive rinse step. Also, during the PBS/BSA rinse, BSA evidently bound to the sensor displacing the remaining His₆-streptavidin; an observation not observed when applying GBP-fusion proteins. The rinse steps here were much more extensive than those for the gold powder assays that indicated very low, but detectable streptavidin binding. Thus, under the conditions used, streptavidin lacking GBP is rather loosely adsorbed to gold surfaces whereas GBP-mediated binding is extremely stable.

The different response to glycerol in FIG. 11 is due to differences in individual sensor operation. Also, the downward drift of the signal for streptavidin lacking GBP may be due to loss of small amounts of protein from the surface during analysis.

### EXAMPLE 6

### Relative Specific Activity of Proteins on Gold Powder

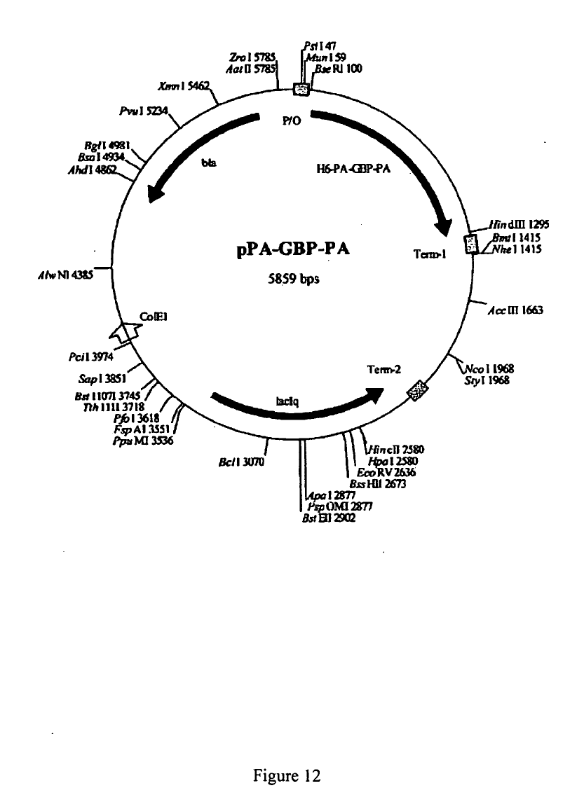

His₆-streptavidin-GBP attached to gold powder bound 5-10 fold more biotinylated antibody than a similar amount of the recombinant His₆-streptavidin based on SDS-PAGE analysis of the extracted protein. The low activity of His₆-streptavidin on gold (see FIGS. 9 and 12) is not a true indication of how much of this protein was adsorbed to gold. The preliminary studies did not carefully quantify the protein concentration. However, the implication is that properly oriented His₆-streptavidin-GBP on gold is much more effective in binding biotinylated molecules than is physically adsorbed His₆-streptavidin. This observation which was not predicted by the prior art indicates that the controlled orientation of GBP-fusion proteins on gold surfaces presents completely accessible binding/active sites resulting in many times more activity than that achieved by physical adsorption or conventional protein chemistry. This is an important benefit achieved by the present invention.

### EXAMPLE 7

### Production of Recombinant Proteins Consisting of a Single Domain of GBP and Multiple Copies of an Individual Fusion Partner

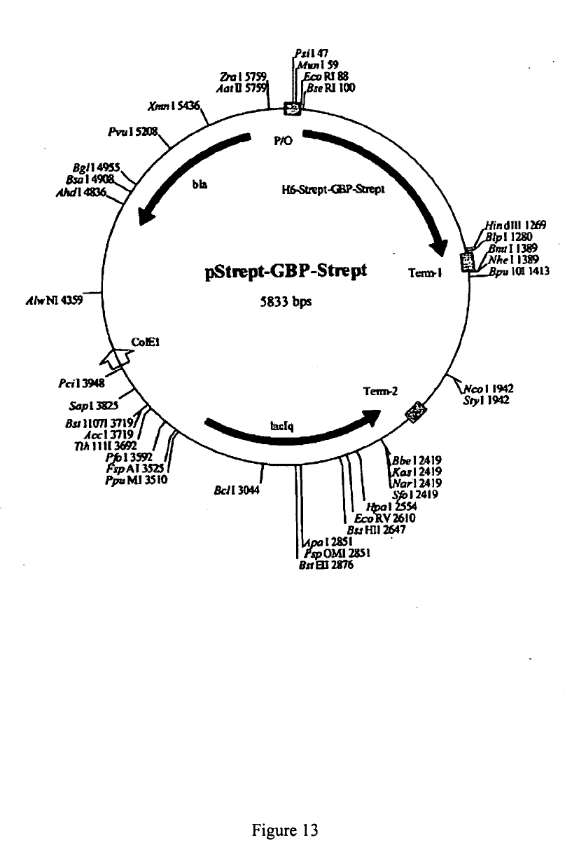

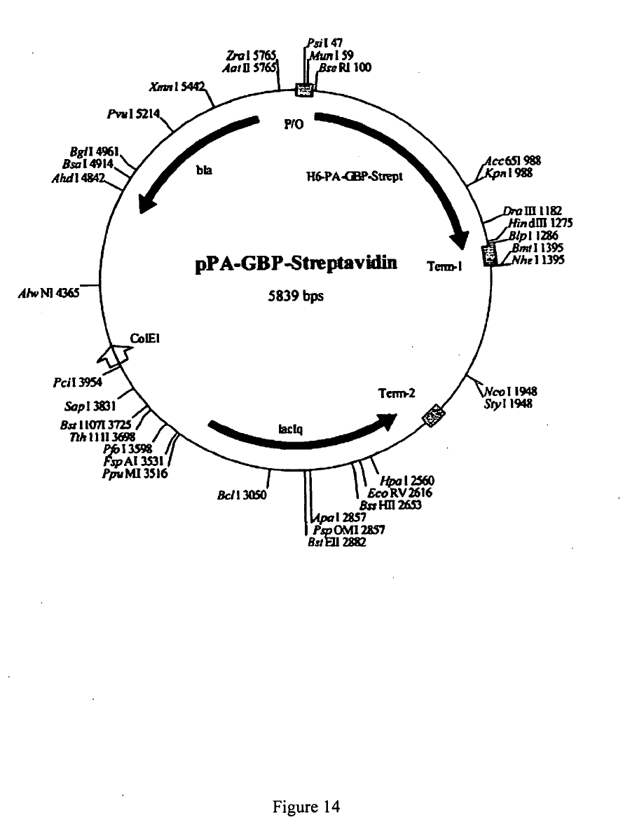

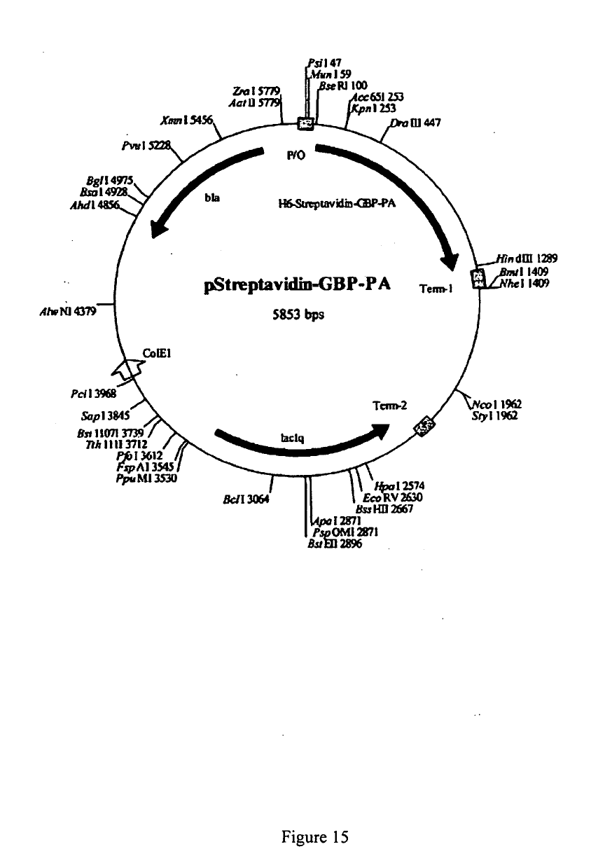

The results presented, herein, in Examples 1 through 6, establish that GBP-fusions can be expressed as stable proteins and rapidly purified with retention of gold-binding and other functions when fusion partners are attached to its amino-terminus of GBP. With the observation that a fusion partner also can be attached to the carboxy-terminus of GBP (Brown, Nat. Biotechnol. 15:269-272, 1997), these observations establish that GBP can accommodate fusion partners at either end of the polypeptide sequence. Consequently, the expression vectors described in Example 1 and depicted in FIG. 1 of this invention can be used to encode a recombinant protein containing a single GBP domain and a minimum of two identical copies of a specific polypeptide fusion partner. Other iterations include: a His₆-protein A-GBP-protein A fusion protein has been expressed in E. coli using the plasmid, pPA-GBP-PA, depicted in FIG. 12, and purified using the His₆ affinity tag; a His₆-streptavidin-GBP-streptavidin fusion protein has been expressed in E coli using the plasmid, pStrept-GBP-Strept, depicted in FIG. 13, and purified using the His₆ affinity tag; a His₆-GBP fusion protein has been expressed in E coli using the plasmid, pGBP, depicted in FIG. 14, and purified using the His₆ affinity tag; a His₆-GBP-GBP fusion protein has been expressed in E coli using the plasmid, pGBP-GBP, depicted in FIG. 15, and purified using the His₆ affinity tag.

Further, as instructed by Example 1, more than one copy of fusion partner linked in tandem at either or both ends of GBP. The presence of flexible linking sequences consisting of glycyl-seryl repeats in the fusion proteins, allows for independent function of each domain of the fusion protein.

Without limiting the scope of the current invention, an example of how multiple copies of a specific fusion partner coupled to GBP can be advantageous relates to the field of biosensors. Biosensors, in general, perform at greater sensitivity with increasing density of recognition molecules, e.g., specific antibody, at the sensing surface. In the specific case of surface plasmon resonance (SPR)-based sensors, the ability to directly detect small analytes in real-time depends on the number of resonance units (RU) that are directly proportional to the density of analyte binding sites at the sensing surface. Similar increases in sensitivity and enhanced performance as illustrated in Example 6 above can be achieved for applications in all fields utilizing gold. Thus, the current invention provides important advantages in overall application performance not provided by existing methods, e.g., random physical adsorption of protein to gold or chemical attachment to foundation layers on gold.

There can be utility in using the recombinant His₆-GBP and His₆-GBP-GBP as agents to block the binding to gold of non targeted substances in test samples following any method to derivatize a gold surface.

### EXAMPLE 8

### Production of Recombinant Proteins Consisting of a Single GBP Domain and at Least One Domain Each of Two Different Fusion Partners

GBP-fusion proteins containing two distinct fusion partners with different function can have broad utility in all fields utilizing gold.

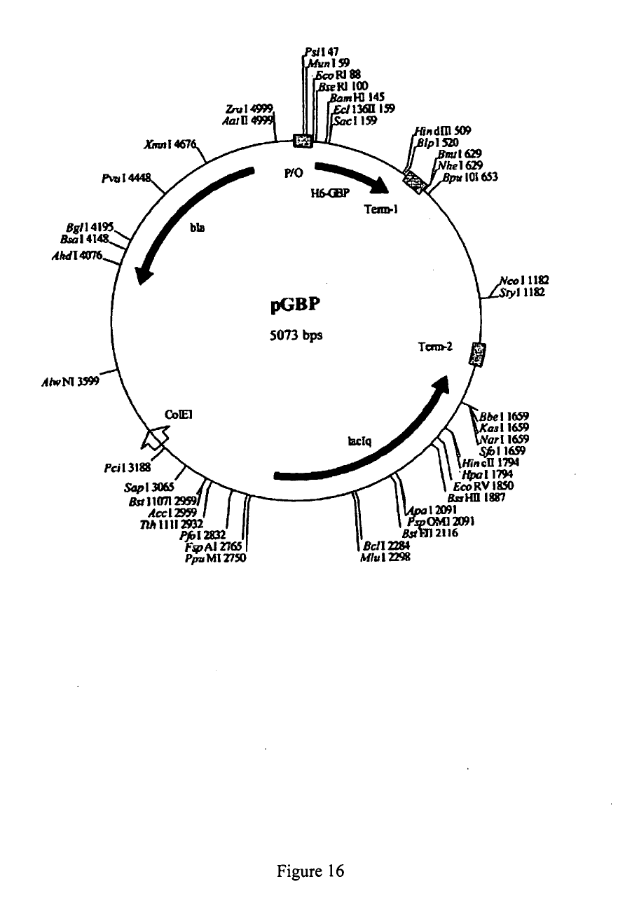

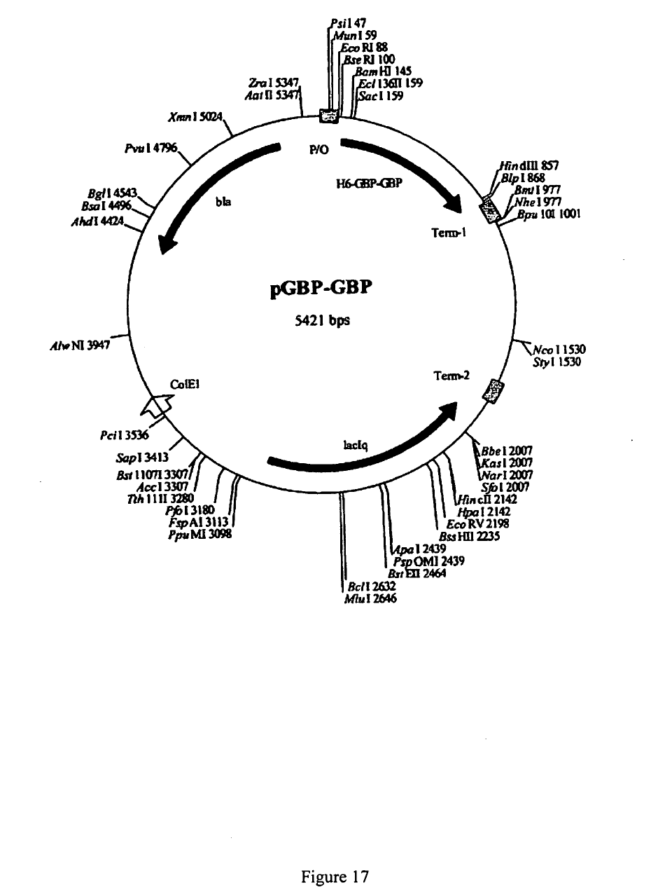

Iterations include: His₆-protein A-GBP-streptavidin fusion protein has been expressed in E. coli using plasmid, pPA-GBP-Streptavidin, as depicted in FIG. 16, and purified using the His₆ affinity tag; His₆-streptavidin-GBP-protein A fusion protein has been expressed in E. coli using plasmid, pStreptavidin-GBP-PA, as depicted in FIG. 17, and purified using the His₆ affinity tag.

Moreover, GBP-fusion partners can be Protein A and other related polypeptides such as Protein G or Protein L or other similar proteins that have immunoglobulin-binding properties distinct from those of Protein A. Such a fusion protein provides the benefit of allowing the detection and binding of more than one class of immunoglobulin simultaneously or sequentially.

The different GBP-fusion partners can be any two polypeptides with distinct affinity binding activity. One advantage of fusion proteins with mixed function as described is to provide versatility by allowing, for example in the case of protein A-GBP-streptavidin, antibody binding activity and any other activity conferred by attachment of biotinylated-molecules, used either sequentially or concurrently. Versatile sensing chips and other surfaces can be constructed using these unique reagents to introduce multiple activities and to achieve improved efficiency and cost reduction compared to the use of existing reagents.

### EXAMPLE 9

### Production of Recombinant Proteins Consisting of Single-Chain Antibodies Fused to GBP

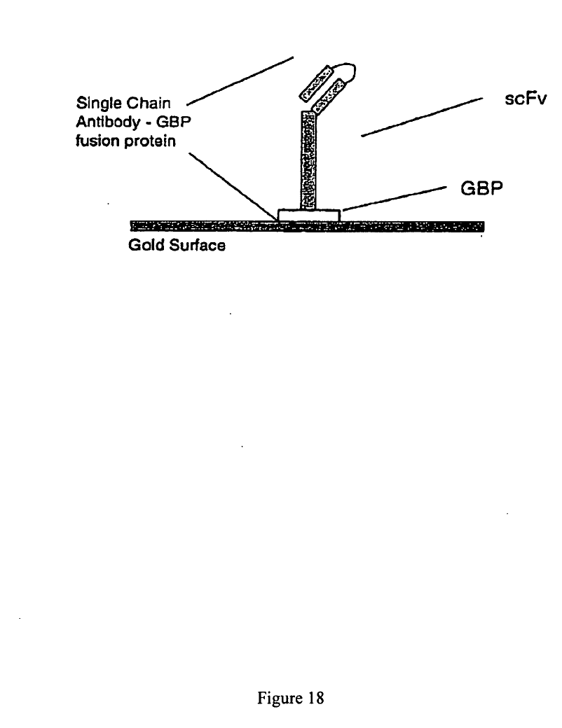

Single chain antibodies (scFvs) consist of variable domains (Fv) separated by linker sequences. Fusion of the scFv construct with different sequences encoding different function has been described. Carboxyl terminal fusion with the gene encoding streptavidin produces an active scFv:streptavidin fusion protein (Kipriyanov, et al., Hum Antibodies Hybridomas 6:93-101, 1995). Cloning of the GBP sequence at the carboxyl terminus of scFv gene sequences produces scFv:GBP fusion constructs which can be expressed in bacteria as described in Example 1 above. Recombinant single-chain antibody fusions produced in this manner can be used to functionalize gold surfaces as illustrated in FIG. 18.

DNA sequences encoding specific single chain antibodies can be obtained by phage selection methods (Clackson, et al., Nature 352:624-628, 1991) or from hybridomas producing monoclonal antibodies. Using the expression plasmids described above in Example 1, those skilled in the art can link the GBP encoding sequence at the C-terminus, or where necessary, at the N-terminus of the sequence encoding scFv antibody. The fusion protein can be expressed, but not limited to, in the cytoplasm of E. coli NovaBlue cells (Novagen) with a His₆-tag at the N-terminus using the QE-80L series of expression vectors (Qiagen). The fusion proteins are likely to accumulate in inclusion bodies and can be purified using a Ni++ column and refolded (Huston, et al., Proc. Natl. Acad. Sci. USA 85:5879-5883, 1988). If necessary, an immuno-affinity purification step can be used to separate out the inactive molecules. If the product is not active, the domains can be shuffled around or periplasmic expression can be employed.

### EXAMPLE 10

### Production of Recombinant Proteins Consisting of an Enzyme, Horse Radish Peroxidase, Fused to GBP

HRP can be expressed in E. coli as inclusion bodies, purified and reconstituted in vitro (Grigorenko, et. al., Biocatalysis and Biotransformation 17:359-379, 1999; Ferapontova, et. al., Biosens Bioelectron 17:953-963, 2002; and Levy, et. al., Biotechnol Bioeng 82:223-231, 2003). Fusion protein constructs can be built as instructed in Example 1 above, placing GBP either at the N-terminus or the C-terminus of HRP with a terminal His₆-tag at the same end as GBP in each case. A flexible linker sequence consisting of glycyl-seryl repeating sequences separates the HRP coding sequence from GBP. These plasmids can be expressed in the cytosol of E. coli, purified from inclusion bodies by Nickel-resin chromatography, and refolded in vitro. The coding sequence for HRP (GenBank Accession # J05552) can be obtained from researchers or synthesized from oligonucleotides.

Further, cytochrome c peroxidase that is also used for derivatizing electrodes (Ruzgas, et. al., Analytica Chimica Acta 330:123-138,1996) can be fused to GBP. The yeast enzyme has been successfully expressed in E. coli (eske, et al., Protein Expr Purif 19:139-147, 2000).

### EXAMPLE 11

### Production of Recombinant Proteins Consisting of an Enzyme, Glucose Oxidase, Fused to GBP

The oxidation of glucose by glucose oxidase and reduction of O2 to H2O2 can provide a measurable electrical current at a nearby conducting electrode, proportional to the concentration of glucose in the sample. Most commercial glucose monitoring devices operate on this principle. However, existing methods for immobilizing glucose oxidase do not allow the sensitivity envisioned for future devices employing nanotechnology designed for non-invasive testing or more accurate testing. Further, the performance of is electrical devices that measure levels of blood glucose can be diminished by irrelevant substances fouling the electrode surface. As established in Examples 4 through 6 above, facilitated protein binding to gold via a GBP domain can significantly improve the attachment of active fusion partners and resist surface fouling compared to conventional methods employing native proteins. The disclosed invention provides similar benefits for the controlled attachment of glucose oxidase in designing improved glucose monitors and in the design of novel miniature nanodevices using gold electrodes for the purpose of detection.

GOx from Aspergillus niger is a dimer of molecular weight 150,000 containing two tightly bound FAD cofactors. It has been extensively used as the basis for biosensors, in glucose detection kits and as a source of hydrogen peroxide in the food industry. It has been expressed and secreted in copious amounts from yeast using either its own signal sequence or the alpha-factor leader sequence of Saccharomyces cerevisiae (Frederick, et al., J Biol Chem 265:3793-3802, 1990, Park, et al., J Biotechnol 81:35-44, 2000). It has also been secreted from S. cerevisiae with a His₆-tag at the C-terminus (Ko, et al., Protein Expr Purif 25:488-493, 2002).

The coding sequence for A. niger GOx (GenBank Accession # J05242) can be obtained from researchers or cloned by PCR from the organism. GBP can be linked to the C-terminus of GOx with a flexible spacer sequence followed by a His₆-tag. Using one of the coli-yeast shuttle vectors (Invitrogen) the fusion protein can be secreted utilizing its own signal sequence according to the method as outlined in Example 1. The host strain of S. cerevisiae carries the appropriate auxotrophic markers for maintaining the plasmid and a pep4 mutation can be used to reduce protein degradation. The fusion protein can be purified from the culture medium using Nickel-resin column chromatography.

The advantage of this yeast expression strategy is that it can produce GOx-GBP in a soluble and active form in large amounts. GBP has numerous serine and threonine residues that could potentially serve as targets for O-linked glycosylation, thus masking gold binding. The electrical communication between GOx and the electrode and thereby its biosensor performance is hampered by the protein-bound carbohydrate moiety of the enzyme (Alvarez-Icaza, et al., Biosens Bioelectron 10:735-742, 1995). A pmr1 host mutation can help in this regard although with an overall inhibition of growth (Ko, et al., Protein Expr Purf 25:488-493, 2002).

To circumvent the potential glycosylation problems mentioned above for yeast-secreted GOx, glucose oxidase from Penicillium amagasakiense can be expressed without carbohydrate in the cytoplasm of E. coli. Further, GOx from P. amagasakiense has a higher turnover rate and a higher affinity for glucose than its A. niger counterpart (Kiess, et al., Eur J Biochem 252:90-99, 1998).

The coding sequence can be cloned by PCR amplification with genomic DNA from the organism as template. GBP-fusion protein constructs can be built placing GBP either at the N-terminus or the C-terminus of GOx with a terminal His₆-tag at the same end as GBP in each case. A flexible linker sequence consisting of glycyl-seryl repeats can separate the GOx gene from GBP. The GOx-GBP fusion proteins can be expressed to form cytoplasmic inclusion bodies and the protein can be purified by Nickel-resin chromatography and subsequently refolded in the presence of FAD cofactor (Witt, et al., Appl Environ Microbiol 64:1405-1411, 1998).

### EXAMPLE 12

### Production of Recombinant Proteins Consisting of GBP, the Enzyme Horseradish Peroxidase, and the Enzyme Glucose Oxidase

The enzymes glucose oxidase and horseradish peroxidase can be used in combination to construct a glucose monitor that has greater sensitivity than one constructed with glucose oxidase alone. Appropriate GBP- and enzyme-containing fusion proteins can provide superior activity in enzyme-based applications compared to available enzymes currently in use.

GBP-fusion partners can be horseradish peroxidase or cytochrome c peroxidase or related peroxidase and glucose oxidase or related enzyme. An advantage of such a fusion protein is to allow a significant increase in the efficiency of activity of each enzyme in enzyme electrodes, e.g., a monitor to measure blood glucose levels. Existing monitoring devices can employ both enzymes in a coupled system to provide enhanced transfer of electrons to an electrode. However, the controlled binding of enzymes provided by the current invention can result in improved efficiency compared to conventional methods to attach enzymes to electrodes.

Appropriate expression vectors can be constructed using methods as described in Example 1.

### EXAMPLE 13

### Production of Recombinant Proteins Consisting of GBP and Cell Surface Receptors or Other Macromolecules; and Production of Recombinant Proteins Consisting of Ligands of Cell Surface Receptors or Other Macromolecules

Fusion proteins consisting of GBP and one or more copies of cell surface receptors or other surface macromolecules can have utility in constructing biodetection devices. In particular, glycosylphosphatidylino-sitol (GPI)-anchored cell surface proteins are widely expressed on the surface of cells, including cells of immunohematopoietic origin. The cross linking via ligand binding of GPI-anchored receptors such as Thy-1, Ly-6 A/E, CD48, CD59 and others induce a variety of T-cell activity including mitogenesis (Loertscher and Lavey, Transpl Immunol 9:93-96, 2002). Such GPI-anchored receptors are the target of intense drug discovery. GPI-anchored proteins do not contain transmembrane amino acid sequences and, therefore, ligand binding and receptor stability is not dependent on the presence of a lipid membrane. Thus, any GPI-linked protein can be a potential fusion partner with GBP for the purpose of defining ligand binding properties and screening for agonists/anatagonists of specific ligands.

### EXAMPLE 15

### Production of Recombinant Proteins Consisting of GBP and a Polypeptide Substrate(S) or a Polypeptide Inhibitor(S) of a Proteolytic Enzyme

Fusion proteins consisting of GBP and certain polypeptide substrates of proteolytic enzymes (proteases) can have utility in clinical and environmental testing. Such fusion proteins can be especially useful when utilized to support biodetection devices designed to detect protease activity in certain physiologic or environmental samples. In many instances, a determination of the presence of protease activity is a tedious process requiring the use of complex analytical equipment.

When used to support biosensors, e.g., SPR devices, fusion proteins of GBP and protease substrates can provide assays to give real-time analysis of protease activity in test samples. Further, such biosensors can function in complex solutions, e.g., crude extracts of tissues or whole blood, where the use of other types of conventional assays including calorimetric, fluorometric, or bio assays are precluded.

Although the invention has been described with reference to the above examples, it will be understood that modifications and variations are encompassed within the spirit and scope of the invention. Accordingly, the invention is limited only by the following claims.

What is claimed is:

1. A device for analyte detection, comprising:
a carrier; and
a first gold-comprising solid phase having a first immobilized fusion protein thereon comprising at least one gold binding protein (GBP) domain and at least one analyte-binding peptide (ABPP) or analyte-binding protein (ABP) domain, wherein the GBP domain comprises SEQ ID NO:1, and wherein the at least one ABPP or ABP is reactive with one or more analytes.

2. The device of claim 1, wherein the first gold-comprising solid phase is a plurality of mobilizable colloidal-gold particles, nano-gold particles, or gold-coated particles comprising a first region on the carrier.

3. The device of claim 2, further comprising a second region on the carrier, wherein the second region comprises at least one immobilized moiety which binds to the at least one ABPP, ABP, analyte, or complexes thereof.

4. The device of claim 3, wherein the carrier comprises:
a first member adapted for drawing a deposited sample from the first region of the carrier to the second region of the carrier, wherein the plurality of mobilizable particles comprise a sample application area and the at least one immobilized moiety comprises a capture zone.

5. The device of claim 2, wherein the first region comprises a wick.

6. The device of claim 5, wherein the wick comprises an absorbent area.

7. The device of claim 3, wherein the immobilized moiety is a peptide, a polypeptide, an organic molecule, an inorganic molecule, a nucleic acid, a lipid, a carbohydrate, a prokaryotic cell, a eukaryotic cell, a virus, or a combination thereof.

8. The device of claim 4, wherein the capture zone precipitates the particles in a detectable pattern, which pattern is a function of the presence or absence of the analyte.

9. The device of claim 4, further comprising a zone intermediate between the first and second region, wherein the intermediate zone comprises an immobilized moiety which binds to mobilized particles containing ABPP or ABP which are unbound by analyte.

10. The device of claim 9, wherein the pattern is detected visually, microscopically, or spectroscopically.

11. The device of claim 1, wherein the device is a test strip.

12. The device of claim 1, wherein the first gold-comprising solid phase is a first gold electrode.

13. The device of claim 12, further comprising a second electrode, wherein the first electrode is a working electrode and the second electrode is a reference electrode.

14. The device of claim 13, further comprising a potentiostat, wherein the poteniostat applies a constant potential to the working electrode.

15. The device of claim 12, wherein the carrier comprises a non-conducting material selected from glass, ceramic, or non-conducting polymers.

16. The device of claim 12, wherein the ABPP or ABP functions as a molecular transducer in the absence of a mediator.

17. The device of claim 16, wherein the fusion protein comprises two or more ABPP or ABP domains.

18. The device of claim 12, wherein the first electrode comprises a second GBP fusion protein, and wherein the at least one ABPP or ABP domain of the first GBP fusion protein is different from the ABPP or ABP domain of the second GBP fusion protein.

19. The device of claim 18, wherein the carrier comprises a surface opposing the first electrode, and wherein the opposing surface comprises a separate immobilized ABPP or ABP.

20. The device of claim 19, wherein the separate immobilized ABPP or ABP reacts with an analyte, a catalytic product of the at least one ABPP or ABP of the first GBP fusion protein, or a substrate of the at least one ABPP or ABP of the first GBP fusion protein.

21. The device of claim 19, wherein the separate immobilized ABPP or ABP generates a catalytic product which interacts with the at least one ABPP or ABP of the first GBP fusion protein.

22. The device of claim 12, wherein a signal is generated upon the reaction of the ABPP or ABP and the analyte via a gain or loss of electrons from the electrode, and wherein the gain or loss of electrons comprises a current flowing in a circuit connected to the first electrode upon the reaction of the ABPP or ABP and the analyte.

23. The device of claim 13, further comprising a third electrode and a first circuit electrically connecting the second and third electrodes for producing a predetermined potential on one of the second and third electrodes, and a second circuit attached to the first electrode whereby a current is produced in the second circuit connected to the first electrode when the ABPP or ABP reacts with the analyte in order to produce a signal proportionate to the concentration of the analyte in a sample.

24. The device of claim 23, wherein the signal is a potential.

25. The device of claim 24, wherein change in potential is measured by a change in impedance.

26. The device of claim 22, further comprising a component for receiving the signal and displaying the corresponding concentration of the analyte.

27. The device of claim 26, further comprising an analog to digital converter that receives the signal and converts the signal to a digital signal.

28. The device of claim 27, further comprising a microprocessor for receiving and processing the digital signal.

29. The device of claim 12, wherein the device is a sensor chip, potentiometric electrode, a piezoelectric quartz sensor, or an amperometric electrode.

30. The device of claim 1, wherein the at least one ABPP or ABP domain is selected from the group consisting of protein A, protein G, streptavidin, core streptavidin, neutravidin, avidin, avidin related protein 4/5, strep-tag, strep-tag II, an antibody, an antibody fragment, a single chain antibody, a protein antigen, a peptide antigen, a peptide toxin, biotin, an enzyme, a receptor, a peptide ligand, a polypeptide substrate, a polypeptide inhibitor, and a combination thereof.

31. The device of claim 30, wherein at least one of the ABPP or ABP domains is an enzyme.

32. The device of claim 31, wherein the enzyme is an oxidase, a oxidoreductase, a hydrolase, an esterase, or a dehydrogenase.

33. The device of claim 32, wherein the enzyme is horseradish peroxidase (HRP), glucose oxidase (GOx), choline esterase, or cholesterol oxidase.

34. The device of claim 1, wherein the analyte is a pesticide, a toxin, a protein, a polypeptide, a hormone, a cytokine, a chemokine, antigen, an antibody, a prokaryotic cell, a eukaryotic cell, a virus, an organic compound, an inorganic compound, a nucleic acid, lipid, a carbohydrate, an ion, an element, or a combination thereof.

35. The device of claim 1, wherein the GBP domain comprises 1 to 7 repeated amino acid sequences as set forth in SEQ ID NO:1.

36. The device of claim 1, wherein the GBP comprises 7 repeated amino acid sequences as set forth in SEQ ID NO:1.

37. The device of claim 1, wherein each domain is separated by one or more peptide linkers of low complexity.

38. The device of claim 37, wherein the linkers comprise at least 5 amino acid residues.

39. The device of claim 37, wherein the linkers are repeating Gly-Ser residues.

40. The device of claim 37, wherein the linkers can be selectively hydrolyzed by enzymes or by chemical reaction.

41. The device of claim 35, wherein binding of GBP to the gold-comprising solid phase is unaffected by substitution of isoleucine for threonine in the fifth position of the last repeated sequence.

42. A method of detecting an analyte comprising:
exposing a device to a sample, an analyte-containing environment, or an analyte containing-surface, wherein the device comprises;
a carrier, and
a first gold-comprising solid phase having a first immobilized fusion protein thereon comprising at least one gold binding protein (GBP) domain and at least one analyte-binding peptide (ABPP) or analyte-binding protein (ABP) domain, wherein the GBP domain comprises SEQ ID NO:1; and
detecting the interaction between the at least one ABPP or ABP and the analyte.

43. The method of claim 42, wherein the first gold-comprising solid phase is a plurality of mobilizable colloidal-gold particles, nano-gold particles, or gold-coated particles comprising a first region on the carrier.

44. The method of claim 43, wherein the device further comprises a second region on the carrier having at least one immobilized moiety which binds to the at least one ABPP or ABP.

45. The method of claim 44, further comprising:
allowing the sample to interact with the mobilizable particles;
immobilizing the particles in at least one capture zone of the second region; and
detecting the presence or absence of the analyte in the at least one capture zone.

46. The method of claim 45, wherein detecting comprises identifying a pattern which is a function of the presence or absence of the analyte.

47. The method of claim 46, wherein when the analyte is present, a first immobilized moiety binds to an ABPP-analyte complex or an ABP-analyte complex.

48. The method of claim 46, wherein when the analyte is absent, a first immobilized moiety binds to an ABPP or an ABP.

49. The method of claim 46, wherein the immobilized moiety is a peptide, a polypeptide, a prokaryotic cell, eukaryotic cell, virus, an organic molecule, an inorganic molecule, a nucleic acid, a lipid, a carbohydrate, or a combination thereof.

50. The method of claim 46, wherein the pattern is detected visually, microscopically, or spectroscopically.

51. The method of claim 44, wherein the analyte is an HIV gp120, or fragment thereof, and the ABPP or ABP is an antibody or fragment thereof which binds to a first epitope of the HIV gp 120 or a fragment thereof.

52. The method of claim 51, wherein the immobilized moiety is an antibody or fragment thereof which binds to a second epitope of the HIV gp120 or a fragment thereof.

53. The method of claim 51, wherein the immobilized moiety binds to the ABPP or ABP in the absence of the HIV gp120 or a fragment thereof.

54. The method of claim 51, wherein the immobilized moiety binds to the ABPP or ABP in the presence of the HIV gp120 or fragment thereof.

55. The method of claim 44, wherein the analyte is an anti-HIV gp120 antibody and the immobilized moiety binds to the ABPP or ABP in the absence of the anti-HIV gp120 antibody or a fragment thereof.

56. The method of claim 55, wherein the immobilized moiety binds to the ABPP or ABP in the presence of the anti-HIV gp120 antibody or fragment thereof.

57. The method of claim 54 or 56, wherein the immobilized moiety binds directly or indirectly to the ABPP or ABP.

58. The method of claim 43, wherein the device is a test strip.

59. The method of claim 42, wherein the first gold-comprising solid phase is a first gold electrode.

60. The method of claim 59, wherein detecting is determined by a signal generated from the interaction.

61. The method of claim 60, wherein the signal is generated upon the reaction of the ABPP or ABP and the analyte via a gain or loss of electrons from the electrode, and wherein the gain or loss of electrons comprises a current flowing in a circuit connected to the first electrode upon the reaction of the ABPP or ABP and the analyte.

62. The method of claim 59, wherein the device further comprises a second electrode, and wherein the first electrode is a working electrode and the second electrode is a reference electrode.

63. The method of claim 62, further comprising a third electrode and a first circuit electrically connecting the second and third electrodes for producing a predetermined potential on one of the second and third electrodes, and a second circuit attached to the first electrode whereby a current is produced in the second circuit connected to the first electrode when the ABPP or ABP reacts with the analyte in order to produce a signal proportionate to the concentration of the analyte in a sample.

64. The method of claim 63, wherein the signal is a potential.

65. The method of claim 64, wherein change in potential is measured by a change in impedance.

66. The method of claim 59, wherein the environment comprises a liquid or a gas.

67. The method of claim 60, further comprising pre-calibrating the device in an environment containing a standard concentration of the analyte.

68. The method of claim 59, wherein the ABPP or ABP functions as a molecular transducer in the absence of a mediator.

69. The method of claim 68, wherein the fusion protein comprises two or more ABPP or ABP domains.

70. The method of claim 59, wherein the first electrode comprises a first and second fusion protein, and wherein the at least one ABPP or ABP domain of the first fusion protein is different from the ABPP or ABP domain of the second fusion protein.

71. The method of claim 59, wherein the carrier comprises a surface opposing the first electrode, and wherein the opposing surface comprises a separate immobilized ABPP or ABP.

72. The method of claim 71, wherein the separate immobilized ABPP or ABP reacts with an analyte, a catalytic product of the at least one ABPP or ABP of the first fusion protein, or a substrate of the at least one ABPP or ABP of the first fusion protein.

73. The method of claim 71, wherein the separate immobilized ABPP or ABP generates a catalytic product which interacts with the at least one ABPP or ABP of the first fusion protein.

74. The method of claim 42, wherein the ABPP or ABP domains are selected from the group consisting of protein A, protein G, streptavidin, core streptavidin, neutravidin, avidin, avidin related protein 4/5, strep-tag, strep-tag II, an antibody, an antibody fragment, a single chain antibody, a protein antigen, a peptide antigen, a peptide toxin, biotin, an enzyme, a receptor, a peptide ligand, a polypeptide substrate, a polypeptide inhibitor, and a combination thereof.

75. The method of claim 74, wherein at least one of the ABPP or ABP domains is an enzyme.

76. The method of claim 75, wherein the enzyme is an oxidase, a oxidoreductase, a hydrolase, an esterase, or a dehydrogenase.

77. The method of claim 76, wherein the enzymes is horseradish peroxidase (HRP), glucose oxidase (GOx), choline esterase, or cholesterol oxidase.

78. The method of claim 59, wherein the device is a sensor chip, potentiometric electrode, a piezoelectric quartz sensor, or an amperometric electrode.

79. The method of claim 42, wherein the GBP domain comprises 1 to 7 repeated amino acid sequences as set forth in SEQ ID NO:1.

80. The method of claim 42, wherein the GBP comprises 7 repeated amino acid sequences as set forth in SEQ ID NO:1.

81. The method of claim 42, wherein each domain is separated by one or more peptide linkers of low complexity.

82. The method of claim 81, wherein the linkers comprise at least 5 amino acid residues.

83. The method of claim 81, wherein the linkers are repeating Gly-Ser residues.

84. The method of claim 81, wherein the linkers can be selectively hydrolyzed by enzymes or by chemical reaction.

85. The method of claim 79, wherein binding of GBP to the gold-comprising solid phase is unaffected by substitution of isoleucine for threonine in the fifth position of the last repeated sequence.

86. The method of claim 42, wherein the analyte is a pesticide, a toxin, a protein, a polypeptide, a hormone, a cytokine, a chemokine, antigen, an antibody, a prokaryotic cell, a eukaryotic cell, a virus, an organic compound, an inorganic compound, a nucleic acid, lipid, carbohydrate, an ion, or an element, or a combination thereof.
